SEQUENCE LISTING (144 sequences)

SEQ ID NO: 1 - DNA, 24 bp, Cannabis spp.
matgthygawvttwgktacrywgm

SEQ ID NO: 2 - DNA, 23 bp, Cannabis spp.
atgthygawvttwgktacrywkm

SEQ ID NO: 3 - DNA, 24 bp, Cannabis spp.
matgtaygaavtttggtacrywgc

SEQ ID NO: 4 - DNA, 24 bp, Cannabis spp.
catgtaygaavtttggtacrywgc

SEQ ID NO: 5 - DNA, 27 bp, Cannabis spp.
atgthygawvttwgktacrywkmthmm

SEQ ID NO: 6 - DNA, 33 bp, Cannabis spp.
gaatmatgthygawvttwgktacrywkmtacct

SEQ ID NO: 7 - DNA, 23 bp, Cannabis sativa
atgtacgaagtttggtacgcagc

SEQ ID NO: 8 - DNA, 23 bp, Artificial Sequence
  THCAS/CBCAS siRNA sense strand sequence
atgtatgaagtttggtacgcagc

SEQ ID NO: 9 - DNA, 23 bp, Artificial Sequence
  THCAS/CBCAS siRNA sense strand sequence
atgtacgaagtttggtacgctgc

SEQ ID NO: 10 - DNA, 23 bp, Cannabis sativa
atgtatgaactttgttacactgc

SEQ ID NO: 11 - DNA, 23 bp, Cannabis sativa
atgtatgatctttggtacactgc

SEQ ID NO: 12 - DNA, 23 bp, Cannabis sativa
atgtttgaactttggtacactgc

SEQ ID NO: 13 - DNA, 23 bp, Cannabis sativa
atgtatgatctttggtacactgc

SEQ ID NO: 14 - DNA, 23 bp, Cannabis sativa
atgtctgaactttggtacactgc

SEQ ID NO: 15 - DNA, 23 bp, Cannabis sativa
atgtatgaacttaggtacactgc

SEQ ID NO: 16 - DNA, 18 bp, Cannabis spp.
atgtatgaaatttggtac

SEQ ID NO: 17 - DNA, 23 bp, Cannabis ruderalis
atgtatgaaatttggtacatagc

SEQ ID NO: 18 - DNA, 19 bp, Cannabis spp.
catgtatgaaatttggtac

SEQ ID NO: 19 - DNA, 24 bp, Cannabis ruderalis
catgtatgaaatttggtacatagc

SEQ ID NO: 20 - DNA, 18 bp, Cannabis spp.
atgtatgaactttggtac

SEQ ID NO: 21 - DNA, 19 bp, Cannabis spp.
aatgtatgaactttggtac

SEQ ID NO: 22 - DNA, 23 bp, Cannabis sativa
atgtatgaactttggtacgctgc

SEQ ID NO: 23 - DNA, 23 bp, Cannabis sativa
atgtatgaactttggtacactga

SEQ ID NO: 24 - DNA, 23 bp, Cannabis spp.
atgtatgaactttggtacactgc

SEQ ID NO: 25 - DNA, 23 bp, Cannabis sativa
atgtatgaactttggtacacttc

SEQ ID NO: 26 - DNA, 19 bp, Cannabis sativa/indica hybrid
catgtatgaactttggtac

SEQ ID NO: 27 - DNA, 24 bp, Artificial Sequence
  THCAS/CBCAS siRNA sense strand sequence
catgtatgaactttggtacactgc

SEQ ID NO: 28 - DNA, 24 bp, Cannabis spp.
aatgtatgaactttggtacactgc

SEQ ID NO: 29 - DNA, 24 bp, Cannabis sativa
catgtacgaagtttggtacgcagc

SEQ ID NO: 30 - DNA, 73 bp, Cannabis spp.
aattmmhktccbtcatcgagckkgaatmatgthygawvttwgktacrywkmthmmtkkkagaagcaagawray

SEQ ID NO: 31 - DNA, 73 bp, Cannabis spp.
aattmmhktccbtcatcgagckkgaatmatgthygawvttwgktacrywgmthmmtkkkagaagcaagaagat

SEQ ID NO: 32 - DNA, 73 bp, Cannabis spp.
aattmmhktccbtcatcgagckkgaatmatgthygawvttwgktacrywgmthmmtkkkagaagcaagataac

SEQ ID NO: 33 - DNA, 45 bp, Cannabis spp.
gagctggaataatgtatgaactttggtacactgctacctgggaga

SEQ ID NO: 34 - DNA, 48 bp, Cannabis spp.
atcgagctggaataatgtatgaactttggtacactgctacctgggaga

SEQ ID NO: 35 - DNA, 54 bp, Cannabis spp.
tccctcatcgagctggaataatgtatgaactttggtacactgctacctgggaga

SEQ ID NO: 36 - DNA, 57 bp, Cannabis spp.
cattccctcatcgagctggaataatgtatgaactttggtacactgctacctgggaga

SEQ ID NO: 37 - DNA, 43 bp, Cannabis spp.
tccattccctcatcgagctggaatcatgtatgaaatttggtac

SEQ ID NO: 38 - DNA, 44 bp, Cannabis sativa/indica hybrid
ttccattccctcatcgagcttgaataatgtatgaactttggtac

SEQ ID NO: 39 - DNA, 44 bp, Cannabis sativa/indica hybrid
ttccattccgtcatcgagctggaatcatgtatgaactttggtac

SEQ ID NO: 40 - DNA, 44 bp, Cannabis spp.
ttccattccctcatcgagctggaataatgtatgaactttggtac

SEQ ID NO: 41 - DNA, 44 bp, Cannabis spp.
ttccattccctcatcgagctggaatcatgtatgaaatttggtac

SEQ ID NO: 42 - DNA, 60 bp, Cannabis sativa/indica hybrid
ttccattccctcatcgagcttgaataatgtatgaactttggtacactgctacctgggaga

SEQ ID NO: 43 - DNA, 60 bp, Cannabis spp.
ttccattccctcatcgagctggaataatgtatgaactttggtacactgctacctgggaga

SEQ ID NO: 44 - DNA, 50 bp, Cannabis sativa
tcatcgagcttgaataatgtatgaactttggtacactgctacctgggaga

SEQ ID NO: 45 - DNA, 50 bp, Cannabis spp.
gagctggaataatgtatgaactttggtacactgctacctgggagaagcaa

SEQ ID NO: 46 - DNA, 51 bp, Cannabis spp.
cgagctggaataatgtatgaactttggtacactgctacctgggagaagcaa

SEQ ID NO: 47 - DNA, 52 bp, Cannabis spp.
tcgagctggaataatgtatgaactttggtacactgctacctgggagaagcaa

SEQ ID NO: 48 - DNA, 53 bp, Cannabis spp.
atcgagctggaataatgtatgaactttggtacactgctacctgggagaagcaa

SEQ ID NO: 49 - DNA, 54 bp, Cannabis spp.
catcgagctggaataatgtatgaactttggtacactgctacctgggagaagcaa

SEQ ID NO: 50 - DNA, 55 bp, Cannabis spp.
tcatcgagcttgaataatgtatgaactttggtacactgctacctgggagaagcaa

SEQ ID NO: 51 - DNA, 55 bp, Cannabis spp.
tcatcgagctggaataatgtatgaactttggtacactgctacctgggagaagcaa

SEQ ID NO: 52 - DNA, 56 bp, Cannabis spp.
ctcatcgagctggaataatgtatgaactttggtacactgctacctgggagaagcaa

SEQ ID NO: 53 - DNA, 56 bp, Cannabis sativa
ctcatcgagctggaataatgtatgaactttggtacacttctacatgggagaagcaa

SEQ ID NO: 54 - DNA, 57 bp, Cannabis spp.
cctcatcgagctggaataatgtatgaactttggtacactgctacctgggagaagcaa

SEQ ID NO: 55 - DNA, 58 bp, Cannabis spp.
ccctcatcgagctggaataatgtatgaactttggtacactgctacctgggagaagcaa

SEQ ID NO: 56 - DNA, 59 bp, Artificial Sequence
  THCAS/CBCAS siRNA sense strand sequence
tccctcatcgagctggaatcatgtacgaagtttggtacgcagctacctgggagaagcaa

SEQ ID NO: 57 - DNA, 59 bp, Cannabis spp.
tccctcatcgagctggaataatgtatgaactttggtacactgctacctgggagaagcaa

SEQ ID NO: 58 - DNA, 61 bp, Cannabis spp.
attccctcatcgagctggaataatgtatgaactttggtacactgctacctgggagaagcaa

SEQ ID NO: 59 - DNA, 62 bp, Cannabis spp.
cattccctcatcgagctggaataatgtatgaactttggtacactgctacctgggagaagcaa

SEQ ID NO: 60 - DNA, 63 bp, Cannabis sativa
ccattccctcatcgagctggaataatgtttgaactttggtacactgctacctgggagaagcaa

SEQ ID NO: 61 - DNA, 63 bp, Cannabis spp.
ccattccctcatcgagctggaataatgtatgaactttggtacactgctacctgggagaagcaa

SEQ ID NO: 62 - DNA, 63 bp, Cannabis sativa
ccattccttcatcgagcttgaataatgtatgaactttggtacactgctacctgggagaagcaa

SEQ ID NO: 63 - DNA, 64 bp, Cannabis spp.
tccattccctcatcgagctggaataatgtatgaactttggtacactgctacctgggagaagcaa

SEQ ID NO: 64 - DNA, 64 bp, Cannabis sativa
tccattccttcatcgagcttgaataatgtatgaactttggtacactgctacctgggagaagcaa

SEQ ID NO: 65 - DNA, 65 bp, Cannabis sativa
ttccattccctcatcgagctggaataatgtttgaactttggtacactgctacctgggagaagcaa

SEQ ID NO: 66 - DNA, 65 bp, Cannabis sativa
ttccattccctcatcgagctggaataatgtatgaactttggtacactgctccctgggagaagcaa

SEQ ID NO: 67 - DNA, 65 bp, Cannabis sativa
ttcctttccctcatcgagctggaatcatgtacgaagtttggtacgcagctacctgggagaagcaa

SEQ ID NO: 68 - DNA, 65 bp, Cannabis sativa
ttcctttccctcatcgagctggaatcatgtacgaagtttggtacgcagctaccttggagaagcaa

SEQ ID NO: 69 - DNA, 65 bp, Cannabis sativa
ttccattccctcatcgagctggaataatgtatgaactttggtacactgctacctggtagaagcaa

SEQ ID NO: 70 - DNA, 65 bp, Cannabis sativa
ttccattccctcatcgagcgggaataatgtatgaactttggtacactgctacctgggagaagcaa

SEQ ID NO: 71 - DNA, 65 bp, Cannabis sativa
ttccattccttcatcgagcttgaataatgtatgaactttgttacactgctacctgggagaagcaa

SEQ ID NO: 72 - DNA, 65 bp, Cannabis sativa
ttccattccttcatcgagcttgaataatgtatgatctttggtacactgctacctgggagaagcaa

SEQ ID NO: 73 - DNA, 65 bp, Cannabis sativa
ttccattccctcatcgagctggaataatgtatgaactttggtacactgataactgggagaagcaa

SEQ ID NO: 74 - DNA, 65 bp, Cannabis sativa
ttcccttccttcatcgagcttgaataatgtctgaactttggtacactgctacctgggagaagcaa

SEQ ID NO: 75 - DNA, 65 bp, Cannabis sativa
ttccattccctcatcgagctggaataatgtatgaacttaggtacactgctacctgggagaagcaa

SEQ ID NO: 76 - DNA, 65 bp, Cannabis sativa
ttccattccttcatcgagcttgaataatgtatgaactttggtacgctgctacctgggagaagcaa

SEQ ID NO: 77 - DNA, 65 bp, Cannabis spp.
ttccattccctcatcgagctggaataatgtatgaactttggtacactgctacctgggagaagcaa

SEQ ID NO: 78 - DNA, 65 bp, Cannabis sativa
ttccattcctacatcgagcttgaataatgtatgaactttggtacactgctacctgggagaagcaa

SEQ ID NO: 79 - DNA, 65 bp, Cannabis sativa
ttacattccctcatcgagctggaataatgtatgaactttggtacactgctacctgtgagaagcaa

SEQ ID NO: 80 - DNA, 65 bp, Cannabis sativa
ttccattccttcatcgagcttgaataatgtatgaactttggtacactgctacctgggagaagcaa

SEQ ID NO: 81 - DNA, 65 bp, Cannabis sativa
ttacattccttcatcgagcttgaataatgtatgaactttggtacactgctacctgggagaagcaa

SEQ ID NO: 82 - DNA, 65 bp, Cannabis sativa
ttcaattccctcatcgagctggaataatgtatgaactttggtacactgcttcctgggagaagcaa

SEQ ID NO: 83 - DNA, 65 bp, Cannabis sativa
ttccagtccctcatcgagctggaataatgtatgaactttggtacactgctacctgggagaagcaa

SEQ ID NO: 84 - DNA, 73 bp, Cannabis spp.
aattccattccctcatcgagctggaataatgtatgaactttggtacactgctacctgggagaagcaagaagat

SEQ ID NO: 85 - DNA, 73 bp, Cannabis sativa
aattccattccctcatcgagctggaataatgtatgaactttggtacactgcttcctgggagaagcaagaagat

SEQ ID NO: 86 - DNA, 65 bp, Cannabis sativa
ttccattccctcatcgagctggaataatgtatgaactttggtacactgcttcctgggagaagcaa

SEQ ID NO: 87 - DNA, 73 bp, Cannabis ruderalis
aattccattccctcatcgagctggaataatgtatgaactttggtacactgctacctgggagaagcaagataac

SEQ ID NO: 88 - DNA, 73 bp, Cannabis ruderalis
aattccattccctcatcgagctggaatcatgtatgaaatttggtacatagcttcatgggagaagcaagaagat

SEQ ID NO: 89 - DNA, 65 bp, Cannabis ruderalis
ttccattccctcatcgagctggaatcatgtatgaaatttggtacatagcttcatgggagaagcaa

SEQ ID NO: 90 - DNA, 1631 bp, Cannabis ruderalis
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggagagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcat

SEQ ID NO: 91 - DNA, 1635 bp, Cannabis ruderalis
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcat

SEQ ID NO: 92 - DNA, 1630 bp, Cannabis ruderalis
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcaaaatggccttgcggctgataatatcattgatgcacacttagtcaatgttgatggaaaagttctagatcgaaaatccatgggagaagatctattttgggctatacgtggtggaggggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcat

SEQ ID NO: 93 - DNA, 1634 bp, Cannabis spp.
atgaattgctcaacattctccttttggttttttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcat

SEQ ID NO: 94 - DNA, 1637 bp, Cannabis ruderalis
atgaattgctcaacattctccttttggtttgtttgcaaaataatatttttctttctctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaaataatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcat

SEQ ID NO: 95 - DNA, 1632 bp, Cannabis ruderalis
atgaagtactcaacattctgtttttggtatgtttgcaagataatatttttctttctctcattcaatatccaaatttcaatagctaatcctcaagaaaacttccttaaatgcttctcacaatatattcccaccaatgtaacaaatgcaaaactcgtatacactcaacacgaccaattttatatgtctatcctaaattcgaccatacaaaatcttagatttacctctgacacaaccccaaaaccacttgttatcatcactcctttaaatgtctcccatatccaaggcactattctatgctccaagaaagttggcttgcagattcgaactcgaagcggtggtcatgatgctgagggcatgtcctacatatctcaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgccctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaacagattgcaaacagttgagctggattgatactatcatcttctacagtggtgttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttaataggttagtaaaagtaaaaaccaaggttgatcccgataatttctttagaaacgaacaaagcatccacctcttcccctgcgtcatcat

SEQ ID NO: 96 - DNA, 1843 bp, Cannabis ruderalis
atttatgtcaattcatttgccccaactccaatatataatattataaataggatagttctctattcataataattcaaaatatcattaggactgaagaaaaatgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggagagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcattaattatctacccatactatgtatgctcccaagtatgaaaatctacattacaactgtgtagactatcataagatatatgtaataaaataaattgtctttcttatttcaatag

SEQ ID NO: 97 - DNA, 1843 bp, Cannabis ruderalis
atttatgtcaattcatttgccccaactccaatatataatattataaataggatagttctctattcataataattcaaaatatcattaggactgaagaaaaatgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcattaattatctacccatactatgtatgctcccaagtatgaaaatctacattacaactgtgtagactatcataagatatatgtaataaaataaattgtctttcttatttca

SEQ ID NO: 98 - DNA, 1843 bp, Cannabis ruderalis
atatttatgtcaattcatttgccccaactccaatatataattataaataggatagttctctattcataataattcaaaatatcattaggactgaagaaaaatgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcaaaatggccttgcggctgataatatcattgatgcacacttagtcaatgttgatggaaaagttctagatcgaaaatccatgggagaagatctattttgggctatacgtggtggaggggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcattaattatctacccatactatgtatgctcccaagtatgaaaatctacattacaactgtgtagactatcataagatatatgtaataaaataaattgtctttcttatttcaatagc

SEQ ID NO: 99 - DNA, 1843 bp, Cannabis ruderalis
atttatgtcaattcatttgccccaactccaatatataatattataaataggatagttctctattcataataattcaaaatatcattaggactgaagaaaaatgaattgctcaacattctccttttggttttttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcattaattatctacccatactatgtatgctcccaagtatgaaaatctacattacaactgtgtagactatcataagatatatgtaataaaataaattgtctttcttatttcaa

SEQ ID NO: 100 - DNA, 1843 bp, Cannabis ruderalis
atttatgtcaattcatttgccccaactccaatatataatattataaataggatagttctctattcataataattcaaaatatcattaggactgaagaaaaatgaattgctcaacattctccttttggtttgtttgcaaaataatatttttctttctctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaaataatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcattaattatctacccatactatgtatgctcccaagtatgaaaatctacattacaactgtgtagactatcataagatatatgtaataaaataaattgtctttcttattt

SEQ ID NO: 101 - DNA, 23 bp, Cannabis spp.
ttgtatgagttatggtacatatg

SEQ ID NO: 102 - DNA, 27 bp, Cannabis spp.
ttgtatgagttatggtacatatgtagy

SEQ ID NO: 103 - DNA, 24 bp, Cannabis spp.
cttgtatgagttatggtacatatg

SEQ ID NO: 104 - DNA, 26 bp, Cannabis spp.
cttgtatgagttatggtacatatgta

SEQ ID NO: 105 - DNA, 27 bp, Cannabis spp.
cttgtatgagttatggtacatatgtag

SEQ ID NO: 106 - DNA, 28 bp, Cannabis spp.
cttgtatgagttatggtacatatgtagy

SEQ ID NO: 107 - DNA, 28 bp, Cannabis spp.
cttgtatgagttatggtacatatgtagc

SEQ ID NO: 108 - DNA, 28 bp, Cannabis sativa
cttgtatgagttatggtacatatgtagt

SEQ ID NO: 109 - DNA, 60 bp, Cannabis spp.
ttccattccctcatcgagctggaatcttgtatgagttatggtacatatgtagttgggaga

SEQ ID NO: 110 - DNA, 61 bp, Cannabis sativa
attccctcatcgagctggaatcttgtatgagttatggtacatatgtagttgggagaagcaa

SEQ ID NO: 111 - DNA, 73 bp, Cannabis sativa
aattccattccctcatcgagctggaatcttgtatgagttatggtacatatgtagttgggagaagcaagaagat

SEQ ID NO: 112 - DNA, 65 bp, Cannabis sativa
ttccattccctcatcgagctggaatcttgtatgagttatggtacatatgtagttgggagaagcaa

SEQ ID NO: 113 - DNA, 65 bp, Cannabis sativa
ttccattccctcatcgagctggaatcttgtatgagttatggtacatatgtagttggtagaagcaa

SEQ ID NO: 114 - DNA, 65 bp, Cannabis sativa
ttccattccctcatcgagatggaatcttgtatgagttatggtacatatgtagttgggagaagcaa

SEQ ID NO: 115 - DNA, 65 bp, Cannabis sativa
ttccagtccctcatcgagctggaatcttgtatgagttatggtacatatgtagttgggagaagcaa

SEQ ID NO: 116 - DNA, 64 bp, Cannabis sativa
tctattccctcatcgagctggaatcttgtatgagttatggtacatatgtagttgggagaagcaa

SEQ ID NO: 117 - DNA, 51 bp, Cannabis spp.
cgagctggaatcttgtatgagttatggtacatatgtagctgggagaagcaa

SEQ ID NO: 118 - DNA, 58 bp, Cannabis spp.
ccctcatcgagctggaatcttgtatgagttatggtacatatgtagctgggagaagcaa

SEQ ID NO: 119 - DNA, 63 bp, Cannabis spp.
ccattccctcatcgagctggaatcttgtatgagttatggtacatatgtagctgggagaagcaa

SEQ ID NO: 120 - DNA, 65 bp, Cannabis spp.
ttccattccctcatcgagctggaatcttgtatgagttatggtacatatgtagctgggagaagcaa

SEQ ID NO: 121 - DNA, 73 bp, Cannabis spp.
aattccattccctcatcgagctggaatcttgtatgagttatggtacatatgtagctgggagaagcaagaagat

SEQ ID NO: 122 - DNA, 73 bp, Cannabis sativa
aattccattccctcattgagctggaatcttgtatgagttatggtacatatgtagctgggagaagcacgaagat

SEQ ID NO: 123 - DNA, 1635 bp, Cannabis ruderalis
atgaagtactcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcagctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagagtcttagtttggctgctgggtattgccctactgtttgcgcaggtggacactttggtggaggaggctatggaccattgatgagaagctatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttctgaatcagcaattccattccctcatcgagctggaatcttgtatgagttatggtacatatgtagctgggagaagcaagaagataacgaaaagcatctaaactggattagaaatatttataacttcatgactccttatgtgtcccaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataatttttttagaaacgaacaaagcatcccacctcttccacggcatcatcattaa

SEQ ID NO: 124 - DNA, 1843 bp, Cannabis ruderalis
atatttagttaatttatttgcccctgctccaatatataaagctataaataggatagttcttaatccatagtaattcaaaattcattagaactaaagaaaaatgaagtactcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcagctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagagtcttagtttggctgctgggtattgccctactgtttgcgcaggtggacactttggtggaggaggctatggaccattgatgagaagctatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttctgaatcagcaattccattccctcatcgagctggaatcttgtatgagttatggtacatatgtagctgggagaagcaagaagataacgaaaagcatctaaactggattagaaatatttataacttcatgactccttatgtgtcccaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataatttttttagaaacgaacaaagcatcccacctcttccacggcatcatcattaatgatcttaaatagatctttttctcttattaattagtccttataatatacatatattgataatatatataaaaaagtttgtccggatgtactgtgtatgctccaatgta

SEQ ID NO: 125 - DNA, 66 bp, Artificial Sequence
  THCAS/CBCAS hpRNA encoding polynucleotide
matgtatgamctttggtacaywgcaaaagtaaagtaatttaagcwrtgtaccaaagktcatacatk

SEQ ID NO: 126 - DNA, 24 bp, Artificial Sequence
  THCAS/CBCAS hpRNA encoding polynucleotdie
matgtatgamctttggtacaywgc

SEQ ID NO: 127 - DNA, 24 bp, Artificial Sequence
  THCAS/CBCAS hpRNA encoding polynucleotide
gcwrtgtaccaaagktcatacatk

SEQ ID NO: 128 - DNA, 66 bp, Artificial Sequence
  THCAS/CBCAS hpRNA encoding polynucleotide
gcwrtgtaccaaagktcatacatkaaaagtaaagtaatttaamatgtatgamctttggtacaywgc

SEQ ID NO: 129 - DNA, 64 bp, Artificial Sequence
  THCAS/CBCAS hpRNA encoding polynucleotide
aataatgtatgaactttggtacaaaaagtaaagtaatttaatgtaccaaagttcatacattatt

SEQ ID NO: 130 - DNA, 74 bp, Artificial Sequence
  THCAS/CBCAS hpRNA encoding polynucleotide
matgtatgamctttggtacaywgcaaaagtaaagtaatttaagcwrtgtaccaaagktcatacatkcctttttt

SEQ ID NO: 131 - DNA, 72 bp, Artificial Sequence
  THCAS/CBCAS hpRNA encoding polynucleotide
atgtatgaaatttggtacatagcaaaagtaaagtaatttaagctatgtaccaaatttcatacatcctttttt

SEQ ID NO: 132 - DNA, 74 bp, Artificial Sequence
  THCAS/CBCAS hpRNA encoding polynucleotide
aatgtatgaactttggtacactgcaaaagtaaagtaatttaagcagtgtaccaaagttcatacattcctttttt

SEQ ID NO: 133 - DNA, 72 bp, Artificial Sequence
  THCAS/CBCAS hpRNA encoding polynucleotide
tacatactttaaaccatgtatcgaaaagtaaagtaatttaacgatacatggtttaaagtatgtacctttttt

SEQ ID NO: 134 - DNA, 74 bp, Artificial Sequence
  THCAS/CBCAS hpRNA encoding polynucleotide
ttacatacttgaaaccatgtgacgaaaagtaaagtaatttaacgtcacatggtttcaagtatgtaacctttttt

SEQ ID NO: 135 - DNA, 74 bp, Artificial Sequence
  THCAS/CBCAS hpRNA encoding polynucleotide
matgtatgaactttggtacactgcaaaagtaaagtaatttaagcagtgtaccaaagttcatacatkcctttttt

SEQ ID NO: 136 - DNA, 80 bp, Artificial Sequence
  THCAS/CBCAS hpRNA encoding polynucleotide
aatgtatgaactttggtacactgctacaaaagtaaagtaatttaagtagcagtgtaccaaagttcatacattcctttttt

SEQ ID NO: 137 - DNA, 127 bp, Artificial Sequence
  THCAS/CBCAS hpRNA encoding polynucleotide
aatgtatgaactttggtacactgcaaaatgtatgaaatttggtacatagcaaaagtaaagtaatttaagctatgtaccaaatttcatacataaaagcagtgtaccaaagttcatacattcctttttt

SEQ ID NO: 138 - DNA, 127 bp, Artificial Sequence
  THCAS/CBCAS hpRNA encoding polynucleotide
aatgtatgaactttggtacactgcaaatacatactttaaaccatgtatcgaaaagtaaagtaatttaacgatacatggtttaaagtatgtaaaaagcagtgtaccaaagttcatacattcctttttt

SEQ ID NO: 139 - DNA, 160 bp, Cannabis sativa
ttgtattgggccggacttttccaacaacaacaaatgcgttaccttgtcaataacggcccacagatttgatttgtacaaagtacaagtctaattgtcccacatcgccaatataacatttccttagagtgtttatatatcgctacctcagtagtttagcttg

SEQ ID NO: 140 - DNA, 287 bp, Artificial Sequence
  Construct comprising THCAS/CBCAS hpRNA encoding polynucleotide operably linked to promoter
ttgtattgggccggacttttccaacaacaacaaatgcgttaccttgtcaataacggcccacagatttgatttgtacaaagtacaagtctaattgtcccacatcgccaatataacatttccttagagtgtttatatatcgctacctcagtagtttagcttgaatgtatgaactttggtacactgcaaaatgtatgaaatttggtacatagcaaaagtaaagtaatttaagctatgtaccaaatttcatacataaaagcagtgtaccaaagttcatacattcctttttt

SEQ ID NO: 141 - RNA, 24 nt, Artificial Sequence
  Illustrative THCAS/CBCAS siRNA stem sequence
aauguaugaacuuugguacacugc

SEQ ID NO: 142 - RNA, 24 nt, Artificial Sequence
  Illustrative THCAS/CBCAS siRNA stem sequence
uuacauacuugaaaccaugugacg

SEQ ID NO: 143 - RNA, 23 nt, Artificial Sequence
  Illustrative THCAS/CBCAS siRNA stem sequence
auguaugaaauuugguacauagc

SEQ ID NO: 144 - RNA, 23 nt, Artificial Sequence
  Illustrative THCAS/CBCAS siRNA stem sequence
uacauacuuuaaaccauguaucg

(19) United States
(12) Patent Application Publication
(10) Pub. No.: US 2023/0357785 A1
(43) Pub. Date: Nov. 9, 2023

(54) GENETICALLY MODIFIED CANNABIS PLANTS WITH NOVEL PHENOTYPES

(71) Applicant: GROWING TOGETHER RESEARCH INC., Fort Wayne, IN (US)

(72) Inventors: C. Michael FRANCIS, Fort Wayne, IN (US); Glen BORCHERT, Fort Wayne, IN (US); Samuel E. PROCTOR, Fort Wayne, IN (US)

(73) Assignee: GROWING TOGETHER RESEARCH INC., Fort Wayne, IN (US)

(21) Appl. No.: 18/027,775

(22) PCT Filed: Sep. 22, 2021
(86) PCT No.: PCT/US2021/051535

§ 371 Date: Mar. 22, 2023

Related U.S. Application Data
(60) Provisional application No. 63/081,900, filed on Sep. 22, 2020.
(60) Provisional application No. 63/188,354, filed on May 13, 2021.

(51) Int. Cl. C12N 15/82; A01H 6/28

(52) U.S. Cl. CPC C12N 15/8243; C12N 15/8218; A01H 6/28

(57) ABSTRACT

This disclosure concerns a genetic platform for obtaining modified Cannabis plant materials exhibiting desirable novel chemotypes and agronomic traits, for example, by modulating the cannabinoid biosynthetic pathway.

### CROSS-REFERENCE TO RELATED APPLICATION

This application claims the benefit of the earlier filing date of, and priority to, U.S. Provisional Application No. 63/081,900, filed on Sep. 22, 2020, and U.S. Provisional Application No. 63/188,354, filed on May 13, 2021, both of which are which incorporated by reference herein in their entirety.

### FIELD OF THE DISCLOSURE

The present disclosure generally relates to genetically modified organisms of Cannabis genus, with at least one altered component of a cannabinoid biosynthetic pathway and/or accessory protein(s). In particular embodiments, genetically modified Cannabis organisms herein exhibit modified and/or improved production of a cannabinoid in one or more cell types and/or tissues. The present disclosure also relates to nucleic acids and methods for the production of such genetically modified Cannabis, to methods for using such genetically modified Cannabis (e.g., to obtain cannabinoids or cannabis commodity products comprising altered cannabinoid phenotypes), and to products obtained therefrom (e.g., flowers, oils, and seed).

### BACKGROUND

Cannabis is a genus of plants that includes a number of species, subspecies, and varieties, examples of which include plants cultivated for fiber and seed production (“fiber types” or “low-intoxicant types,” also sometimes referred to as “hemp”), plants cultivated for bioactive chemical production (“high-intoxicant types”), and escaped, hybridized, or wild forms of the foregoing. Organisms of the Cannabis genus are diploid plants (2n=20) for which sometimes conflicting genomic data has been published, purporting to cover the entire genome. van Bakel et al., Gen. Biol. 12(10):R102, 2011; Kojoma et al., Forens. Sci. Int. 159:132-40, 2006; Laverty et al., Genom. Res. 29:146-56, 2019. Due to the classification of high-intoxicant cannabis as a controlled substance under the Controlled Substances Act, and similar controlled substances regimes in other countries, cannabis research and breeding has lagged behind that of other cultivated plants. Accordingly, there are myriad unaddressed agronomic issues arising during the cultivation of cannabis, such as vulnerability to damage from insects and fungal diseases, and flowering requirements that present significant obstacles to the grower, particularly in suboptimal growing conditions.

Cannabis produces a number of useful chemical compounds, including cannabinoids and terpenoids, which are secreted by glandular trichomes that occur mostly on floral calyxes and bracts of female plants. Cannabinoids are a complex group of chemicals, of which over 113 have been identified. ElSohly & Slade, Life Sci. 78:539-48, 2005. The phytocannabinoids found in naturally-occurring cannabis plants include C₂₁ terpenophenols, such as tetrahydrocannabinol (THC) and cannabidiol (CBD). Cannabis plants are conventionally classified as being high-intoxicant or low-intoxicant based on the relative proportion of THC to CBD, but types grown for psychoactive use generally produce large amounts of both. In addition to THC and CBD, the class of known phytocannabinoids includes cannabinol (CBN), cannabigerol (CBG), cannabichromene (CBC), cannabicyclol (CBL), cannabivarin (CBV), cannabielsoin (CBE), cannabicitran (CBT), and derivatives of the foregoing. Cannabinoids exist in the plant in acidic precursor forms. Heat, light, or alkaline conditions lead to decarboxylation of these acidic cannabinoid precursors (e.g. tetrahydrocannibinolic acid (THCA) is decarboxylated to form THC). The pharmacological effects of cannabinoids are thought to be predominantly through their action on CB1 and CB2 receptors, where behavioral and metabolic actions are generally assigned to CB1, and immune response modulation generally assigned to CB2.

Cannabinoids are synthesized in the wild-type plant from CBG-type compounds, often differing in the way in which CBG is cyclized. The first enzyme in the cannabinoid biosynthetic pathway is hexanoyl-CoA synthetase (AAE1), which produces hexanoyl-CoA as a substrate from which tetraketide synthase and olivetolic acid cyclase synthesize olivetolic acid. Olivetolic acid is geranylated by an aromatic prenyltransferase enzyme to form the branch-point intermediate cannabigerolic acid (CBGA). Through the action of oxidocyclase enzymes that are not well-understood, CBGA is converted to acidic cannabinoids, for example, THCA, CBDA, and CBCA.

Cannabinoid biosynthesis genes, including THCA synthase (THCAS), CBDA synthase (CBDAS), and CBCA synthase (CBCAS), are generally unlinked. Laverty et al. (2019). Loci for THCAS and CBDAS are found within retrotransposon-rich regions of −40 Mb of minimally recombining repetitive DNA that are highly nonhomologous between high-intoxicant and low-intoxicant plant alleles, and have likely evolved through extensive chromosomal rearrangement and gene duplication events. THCAS and CBDAS may be allelic at the same locus, but most cannabinoid biosynthesis genes other than THCAS and CBDAS are randomly distributed across the genome, including at least one copy of AAE1, tandem copies of tetraketide synthase and olivetolic acid cyclase, and at least three copies of CBCAS. This complicated genetic map is markedly different than what is found in many cultivated crops, where genes of biosynthetic pathways often occur in gene clusters that simplify the process of trait introgression. Further complicating the analysis of cannabinoid synthases is the fact that particular clones sequences are often incorrectly annotated in databases.

US Patent Publication No. 2014/0057251 A1 reports sequence assembly data from high-intoxicant C. sativa and C. indica, including variable partially overlapping sequences for several cannabinoid synthase genes from these plants. US Patent Publication No. 2018/0258439 A1 proposes the modulation of endogenous phytocannabinoids in Cannabis by inhibition of a THCAS gene, for example, by RNA interference. U.S. Pat. No. 10,364,416 relates to decreasing cannabinoid production in organisms by modifying the expression of one CBCAS gene.

Despite suggestions and outlines of strategies in the prior art for modification of THCA, CBDA, and/or CBCA levels in Cannabis sativa in this very active field through the genetic modification of THCAS or CBCAS (see U.S. Pat. No. 10,364,416), no such modification has successfully been produced to the knowledge of the Applicant.

### BRIEF SUMMARY OF ASPECTS OF THE DISCLOSURE

Disclosed herein are genetically modified Cannabis plant materials (e.g., viable plants, explants, and seed) and commodity products derived therefrom, wherein the genetically modified Cannabis plant materials exhibits a novel phenotype; for example, a modified chemotype (e.g., modified cannabinoid expression and/or content). Some embodiments herein include a genetically modified Cannabis plant comprising a heterologous polynucleotide encoding an iRNA molecule that decreases or effectively silences the expression of one or more cannabinoid biosynthetic genes; for example, THCAS/CBCAS. In particular embodiments herein, the heterologous polynucleotide encodes an iRNA molecule that decreases or effectively silences the expression of every genomic copy of THCAS/CBCAS, without significantly affecting the expression of CBDAS. In particular examples, a genetically modified THC-null Cannabis plant material (e.g., a transgenic plant) is provided that may be used, for example, as an engineering platform for the production of a cannabinoid other than THC/THCA, or the modulation of the relative amounts of cannabinoids other than THC/THCA in the plant. In these and further examples, a genetically modified CBC-null Cannabis plant material (e.g., a THC-null, CBC-null Cannabis plant material) is provided that may be used, for example, as an engineering platform for the production of a cannabinoid other than CBC/CBCA, or the modulation of the relative amounts of cannabinoids other than CBC/CBCA in the plant. Expressing the hpRNA molecules of certain embodiments herein in a Cannabis plant (hemp-types or high-intoxicant types) substantially eliminates or reduces THCA and CBCA, for example, to undetectable levels. In particular embodiments, the elimination of the biosynthetic pathway from CBGA to THCA and CBCA results in an increase in CBDA/CBD production, and in some examples, the presence or an increased amount of other products; e.g., cannabinol (CBN), cannabigerol (CBG), cannabichromene (CBC), cannabicyclol (CBL), cannabivarin (CBV), cannabielsoin (CBE), cannabicitran (CBT), and derivatives of the foregoing.

Accordingly, the present disclosure provides nucleic acid molecules (e.g., DNAs, dsRNAs, siRNAs, miRNAs, shRNAs, and hpRNAs), and methods of use thereof, for the silencing of at least one cannabinoid synthase. Particular embodiments herein provide an hpRNA that is effective for inhibiting or essentially silencing a plurality of THCAS/CBCAS gene copies in Cannabis. The presence of multiple copies of these highly related genes in the cannabis genome complicates the targeting of these genes, as homologous recombination will restore gene expression if single sequences are targeted by RNAi (RNA interference) or CRISPR. Particular embodiments herein include a polynucleotide expressing hpRNAs that are engineered to include an siRNA that targets a particular highly homologous sequence motif found in the THCAS/CBCAS and CBDAS mRNA. When introduced into Cannabis plant materials, the presence of characteristic differences in sequence between the motif in THCAS/CBCAS and CBDAS allows these genes to be selectively inhibited. To be clear, the homologous motif sequence difference is not seen between THCAS and CBCAS, which enables targeting both of them simultaneously with the same hpRNA. For example, variants of the conserved motif may be targeted to selectively knockout all copies of THCAS/CBCAS in the genome, thereby preventing reactivation of the genes by recombination.

Specifically disclosed are polynucleotides that encode a hpRNA molecule targeting the highly homologous THCAS and/or CBCAS sequence motif, for example, to inhibit or essentially silence a plurality of THCAS/CBCAS genes in a Cannabis plant material. Some embodiments include a DNA molecule comprising a polynucleotide encoding a hpRNA molecule in an expression cassette. For example, a DNA molecule may comprise the polynucleotide operably linked to one or more regulatory elements; for example, a promoter (e.g., a plant promoter operable in the Cannabis plant). In methods according to some embodiments, a Cannabis plant, plant part, plant tissue, plant tissue culture, plant cell, or plant cell culture may be transformed with such a DNA molecule to produce a genetically modified Cannabis material. In these and further embodiments, the genetically modified Cannabis plant material may be regenerated to produce a transgenic Cannabis plant, for example, with a modified chemotype with respect to the wild-type plant. Therefore, some embodiments herein provide a genetically modified Cannabis plant comprising a polynucleotide encoding a hpRNA molecule targeting the highly homologous THCAS/CBCAS sequence motif. Identification of such a polynucleotide in a Cannabis plant material indicates that the plant or plant material is a genetically modified Cannabis plant material of the present disclosure.

Also described herein are cannabis commodity products obtained from a genetically modified Cannabis plant material comprising a polynucleotide that encodes a hpRNA molecule targeting the highly homologous THCAS and/or CBCAS sequence motif, wherein the commodity product comprises the polynucleotide, even if at a trace or insignificant amount (e.g., oil that comprises predominately cannabinoid(s)). Such commodity products benefit from the novel chemotype contributed by the polynucleotide and the hpRNA encoded therefrom. A cannabis commodity product herein may be a medical product having an increased amount of CBD or a specific desirable ratio of CBD:CBG:CBN, or a recreational product (for example, having a ratio of cannabinoids desirable for recreational consumption, and optionally a higher level of THC). In particular embodiments, the cannabis commodity product is selected from the group consisting of food products (for example, baked goods (e.g., cookies), beverages (e.g., coffee and soda), candy, and consumable oils, extracts, and concentrates), inhalable products (e.g., cigarettes and vape oils), concentrates (e.g., for use in vaporizers), creams (e.g., face creams and tattoo creams), extracts, flower, hemp, fiber, oils (e.g., body oils, beard oils, and massage oils), medicaments (e.g., Epidiolex™ and Sativex™), salves, ointments, cosmetics, soaps, lip balms, hair products (e.g., shampoos), bath bombs, bath salts, gels (e.g., topical gels), lotions, roll-on skin products and deodorants, patches (e.g., topical patches and transdermal patches), capsules, tablets, strips (e.g., oral, dissolving strips), and any of the foregoing, formulated for human use or use by animals (e.g., pets).

Further described are means for producing a THC-null/CBC-null Cannabis plant. Means for producing a THC-null/CBC-null Cannabis plant include a polynucleotide characterized by SEQ ID NO: 145. Some embodiments herein include a Cannabis plant comprising a means for producing a THC-null/CBC-null Cannabis plant. Some embodiments include a cannabis commodity product comprising a means for producing a THC-null/CBC-null Cannabis plant.

Another embodiment is a nucleic acid molecule including at least one polynucleotide operably linked to a plant promoter that functions in a Cannabis plant, wherein the polynucleotide encodes a hairpin RNA (hpRNA) molecule, and wherein the polynucleotide includes: a first nucleotide sequence encoding a first polyribonucleotide in the hpRNA molecule, wherein the first nucleotide sequence is between 20 and 30 nucleotides in length and is substantially identical to the complement or reverse complement of a Cannabis THCAS/CBCAS gene, and the first nucleotide sequence includes at least 12 contiguous nucleotides of the complement or reverse complement of SEQ ID NO: 17 or SEQ ID NO: 28, and a second nucleotide sequence encoding a sense polyribonucleotide in the hpRNA molecule that is substantially the reverse complement of the first nucleotide sequence, wherein the first and second nucleotide sequence are separated in the polynucleotide by a nucleotide sequence that encodes a loop structure in the hpRNA molecule.

Also provided is a nucleic acid molecule including at least one polynucleotide operably linked to a plant promoter that functions in a Cannabis plant, wherein the polynucleotide encodes a hairpin RNA (hpRNA) molecule, and wherein the polynucleotide includes: a first nucleotide sequence encoding a first polyribonucleotide in the hpRNA molecule, wherein the first nucleotide sequence is between 20 and 30 nucleotides in length and is substantially identical to the complement or reverse complement of a Cannabis THCAS/CBCAS gene, and is selected from the group consisting of: a nucleotide sequence that includes at least 12 contiguous nucleotides of the complement or reverse complement of the motif defined by SEQ ID NO: 1 and SEQ ID NOs: 2-29, preferably wherein the nucleotide sequence includes at least 12 contiguous nucleotides of the complement or reverse complement of a nucleotide sequence selected from the group consisting of SEQ ID NOs: 17, 19, 24, 28, 43, and 80, most preferably wherein the nucleotide sequence includes at least 12 contiguous nucleotides of the complement or reverse complement of SEQ ID NO: 17 or SEQ ID NO: 28, and a nucleotide sequence that is at least 80% identical over its length to the complement or reverse complement of the motif defined by SEQ ID NO: 1 and SEQ ID NOs: 2-29, preferably wherein the nucleotide sequence is at least 80% identical over its length to the complement or reverse complement of a nucleotide sequence selected from the group consisting of SEQ ID NOs: 17, 19, 24, 28, 43, and 80, most preferably wherein the nucleotide sequence is at least 80% identical over its length to the complement or reverse complement of SEQ ID NO: 17 or SEQ ID NO: 28, even more preferably wherein the nucleotide sequence is at least 90% identical over its length to the complement or reverse complement of the motif defined by SEQ ID NO: 1 and SEQ ID NOs: 2-29, preferably wherein the nucleotide sequence is at least 90% identical over its length to the complement or reverse complement of a nucleotide sequence selected from the group consisting of SEQ ID NOs: 17, 19, 24, 28, 43, and 80, most preferably wherein the nucleotide sequence is at least 90% identical over its length to the complement or reverse complement of SEQ ID NO: 17 or SEQ ID NO: 28; a second nucleotide sequence encoding a sense polyribonucleotide in the hpRNA molecule that is substantially the reverse complement of the first nucleotide sequence, wherein the first and second nucleotide sequence are separated in the polynucleotide by a nucleotide sequence that encodes a loop structure in the hpRNA molecule.

Also provided is nucleic acid molecule including at least one polynucleotide operably linked to a plant promoter that functions in a Cannabis plant, wherein the polynucleotide encodes a hairpin RNA (hpRNA) molecule, and wherein the polynucleotide includes: a first nucleotide sequence encoding a first polyribonucleotide in the hpRNA molecule, wherein the first nucleotide sequence is between 20 and 30 nucleotides in length and is substantially identical to the complement or reverse complement of a Cannabis THCAS/CBCAS gene, and is selected from the group consisting of: a nucleotide sequence that includes at least 12 contiguous nucleotides of the complement or reverse complement of the motif defined by SEQ ID NO: 1 and SEQ ID NOs: 2-29, and a nucleotide sequence that is at least 80% identical over its length to the complement or reverse complement of the motif defined by SEQ ID NO: 1 and SEQ ID NOs: 2-29, a nucleotide sequence is at least 90% identical over its length to the complement or reverse complement of the motif defined by SEQ ID NO: 1 and SEQ ID NOs: 2-29; a second nucleotide sequence encoding a sense polyribonucleotide in the hpRNA molecule that is substantially the reverse complement of the first nucleotide sequence, wherein the first and second nucleotide sequence are separated in the polynucleotide by a nucleotide sequence that encodes a loop structure in the hpRNA molecule.

Another embodiment is a nucleic acid molecule including at least one polynucleotide operably linked to a plant promoter that functions in a Cannabis plant, wherein the polynucleotide encodes a hairpin RNA (hpRNA) molecule, and wherein the polynucleotide includes: a first nucleotide sequence encoding a first polyribonucleotide in the hpRNA molecule, wherein the first nucleotide sequence is between 20 and 30 nucleotides in length and is substantially identical to the complement or reverse complement of a Cannabis CBDAS gene, and the first nucleotide sequence includes at least 12 contiguous nucleotides of the complement or reverse complement of SEQ ID NO: 103, and a second nucleotide sequence encoding a sense polyribonucleotide in the hpRNA molecule that is substantially the reverse complement of the first nucleotide sequence, wherein the first and second nucleotide sequence are separated in the polynucleotide by a nucleotide sequence that encodes a loop structure in the hpRNA molecule.

Also described is a nucleic acid molecule including at least one polynucleotide operably linked to a plant promoter that functions in a Cannabis plant, wherein the polynucleotide encodes a hairpin RNA (hpRNA) molecule, and wherein the polynucleotide includes: a first nucleotide sequence encoding a first polyribonucleotide in the hpRNA molecule, wherein the first nucleotide sequence is between 20 and 30 nucleotides in length and is substantially identical to the complement or reverse complement of a Cannabis CBDAS gene, and is selected from the group consisting of: a nucleotide sequence that includes at least 12 contiguous nucleotides of the complement or reverse complement of the motif defined by SEQ ID NO: 103, and a nucleotide sequence that is at least 80% identical over its length to the complement or reverse complement of the motif defined by SEQ ID NO: 103, even more preferably wherein the nucleotide sequence is at least 90% identical over its length to the complement or reverse complement of the motif defined by SEQ ID NO: 103; a second nucleotide sequence encoding a sense polyribonucleotide in the hpRNA molecule that is substantially the reverse complement of the first nucleotide sequence, wherein the first and second nucleotide sequence are separated in the polynucleotide by a nucleotide sequence that encodes a loop structure in the hpRNA molecule.

Further embodiments are genetically modified Cannabis plant material and commodity products made of or from a Cannabis plant (e.g., C. sativa, C. indica (non-hybrid), a C. sativa/C. indica hybrid, or C. sativa subspecies, C. ruderalis) including the polynucleotide from the a nucleic acid molecule or a hpRNA molecule encoded by the polynucleotide described herein.

Also provided are vectors configured to express the nucleic acid molecule of any one of the provided embodiments; and cells derived from the genetically modified Cannabis plant described herein, which cell includes a hpRNA molecule described herein or a nucleic acid molecule encoding it.

Yet another embodiment is a method of making a genetically modified Cannabis plant, including: transforming a cell of a Cannabis plant with the vector to produce a transformed cell; regenerating a plant from the transformed cell, which plant is the genetically modified Cannabis plant.

Also described are method of modifying a Cannabis plant using a CRISPR/Cas9 system, substantially as described herein. The CRISPR/Cas9 system is in some embodiments used to modify Cannabis THCAS/CBCAS gene(s) or to modify Cannabis CBDAS gene(s). Such CRISPR/Cas9 system in some instances targets the THCAS/CBCAS genes using a consensus sequence described herein; and in some instances, it targets a CBDAS consensus sequence describe herein.

The foregoing and other features will become more apparent from the following detailed description of several embodiments, which proceeds with reference to the accompanying Figures.

### BRIEF DESCRIPTION OF THE FIGURES

FIG. 1 includes a diagram of the cannabinoid biosynthesis pathway, showing the conversion of CBGA into THCA, CBCA, and CBDA by THCAS, CBCAS, and CBDAS, respectively.

FIGS. 2A-2B include an illustration of a process by which the nucleotide sequence motif described herein in cannabinoid synthase genes can be used to overcome obstacles to stably modifying cannabinoid-related traits in Cannabis.

FIG. 2A includes an alignment of a highly homologous nucleotide sequence motif (SEQ ID NO: 1) in all twelve genomic copies of the Cannabis THCAS/CBCAS genes with the corresponding motif (SEQ ID NO: 103) in the single genomic copy of Cannabis CBDAS, which is extremely highly conserved across species of the genus. Specific nucleotides that diverge between Cannabis THCAS/CBCAS and Cannabis CBDAS are shown in bold font.

FIG. 2B illustrates rational siRNA design for specific inhibition of all twelve THCAS/CBCAS genes. In the uppermost panel, the Cannabis THCAS/CBCAS motif (SEQ ID NO: 1) is presented above representative sequences from THCAS/CBCAS in C. sativa, C. sativa/indica, and C. ruderalis (see FIGS. 3A-3B). siRNAs sequences of 23-24 nucleotides in length are shown, but it is known in the art that siRNAs of shorter or longer lengths may be used. It is further known in the art that a 23-24 nucleotide siRNA sequence can tolerate up to two mismatches with its target while retaining function. A first nucleotide sequence (SEQ ID NO: 17) is shown immediately beneath the THCAS/CBCAS motif (SEQ ID NO: 1). This sequence matches the motif from a C. ruderalis (Finola) THCAS/CBCAS sequence (SEQ ID NO: 89), and the complement and reverse complement of this first nucleotide sequence encode siRNAs that are effective inhibitors of THCAS/CBCAS gene targets such as those comprising SEQ ID NO: 89, but SEQ ID NO: 17 is mismatched at three positions (shown with hashes) with motifs from other THCAS/CBCAS gene sequences from C. ruderalis (Finola) and C. sativa (SEQ ID NO: 77), and from C. sativa (SEQ ID NO: 80).

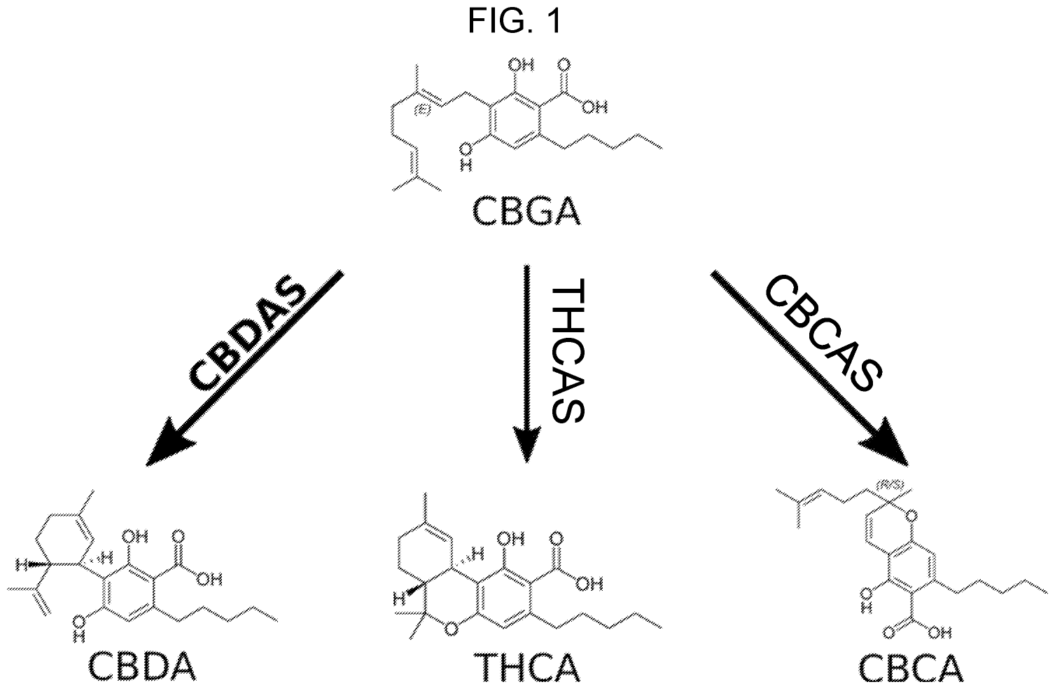

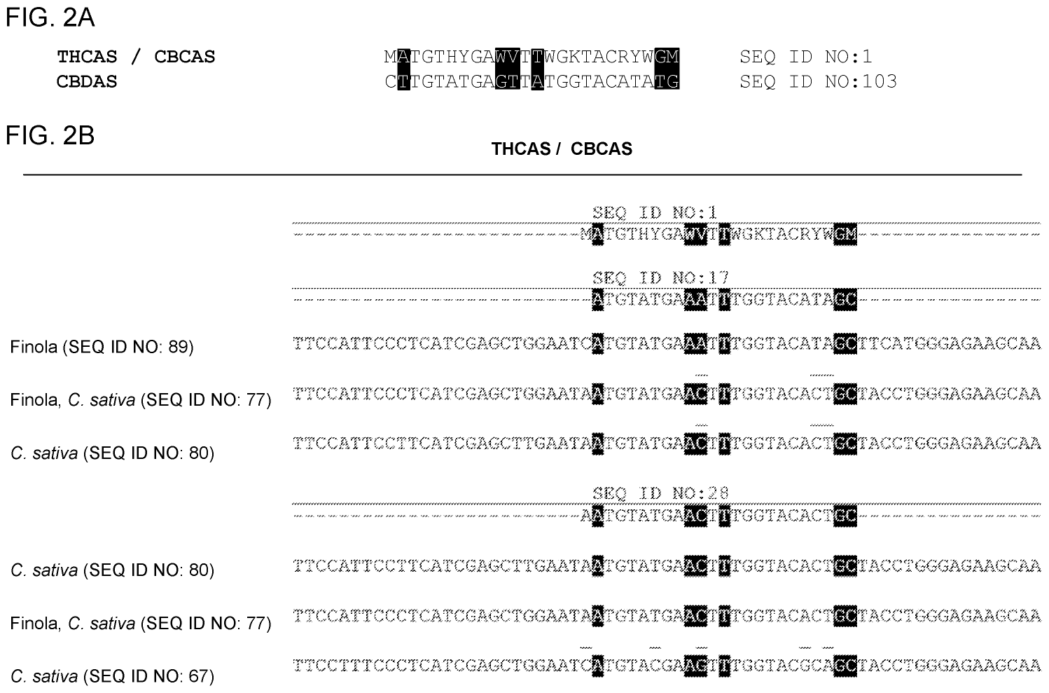

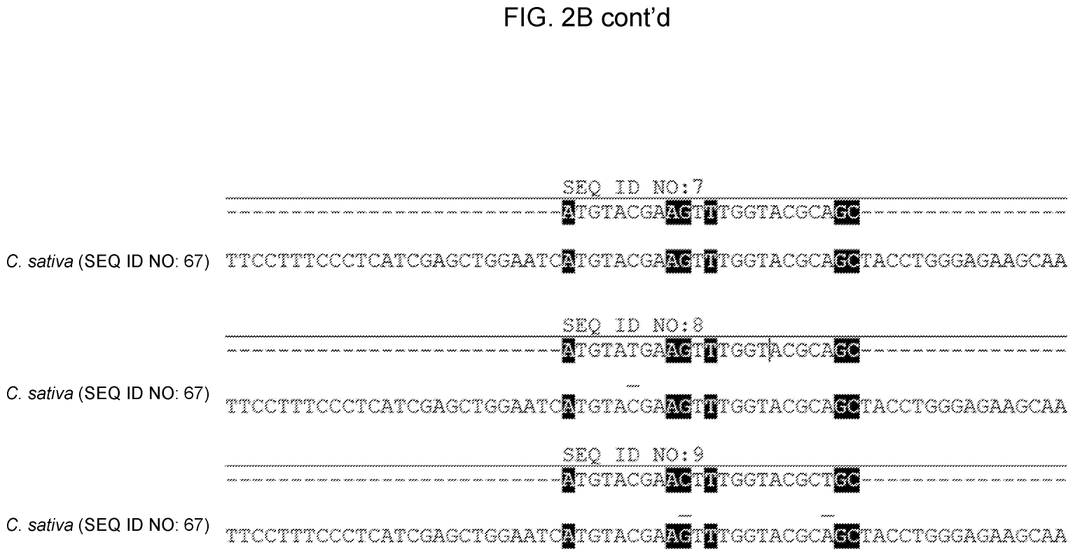

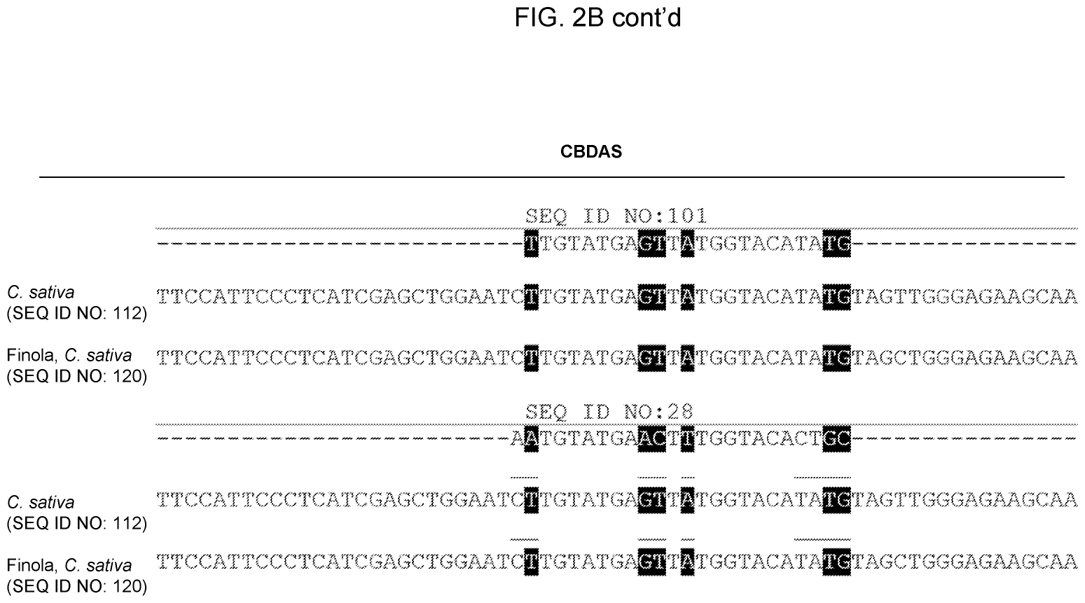

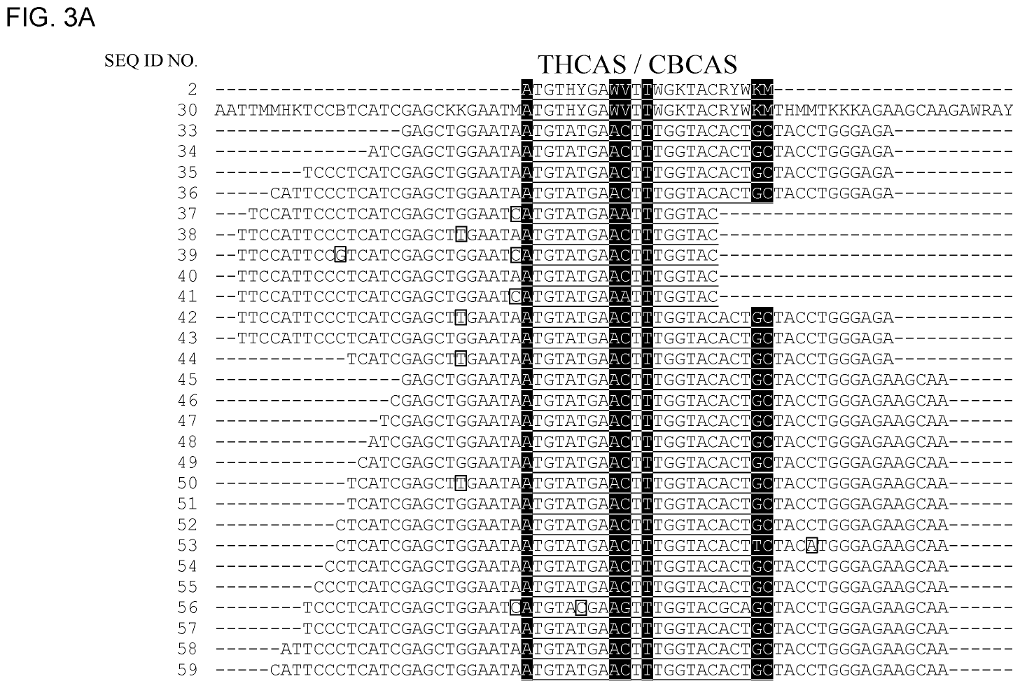

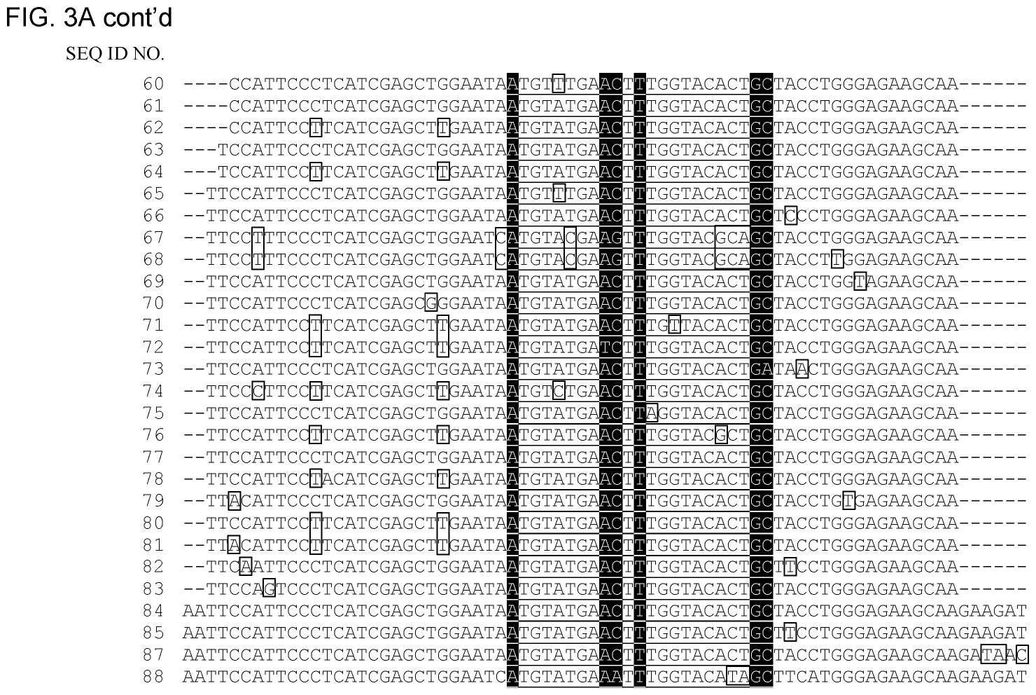

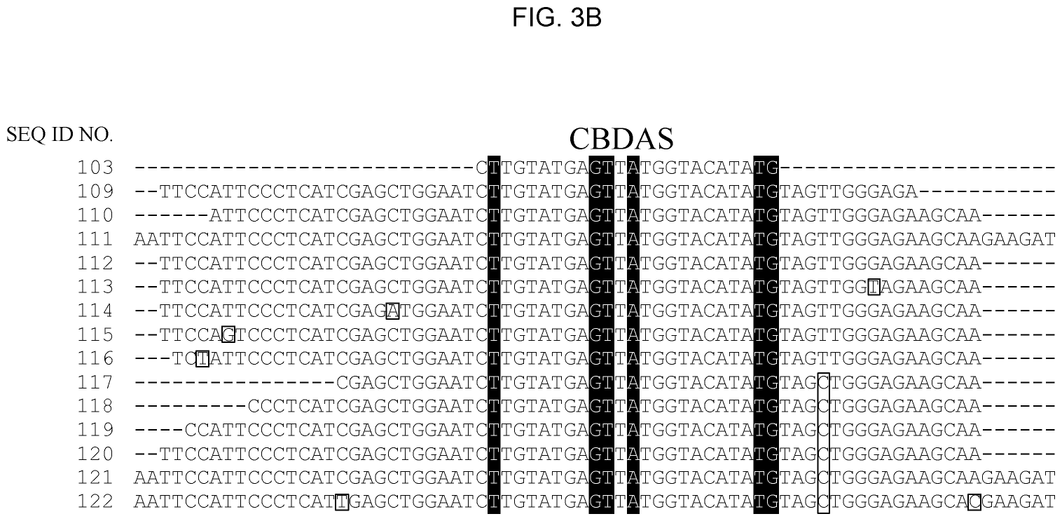

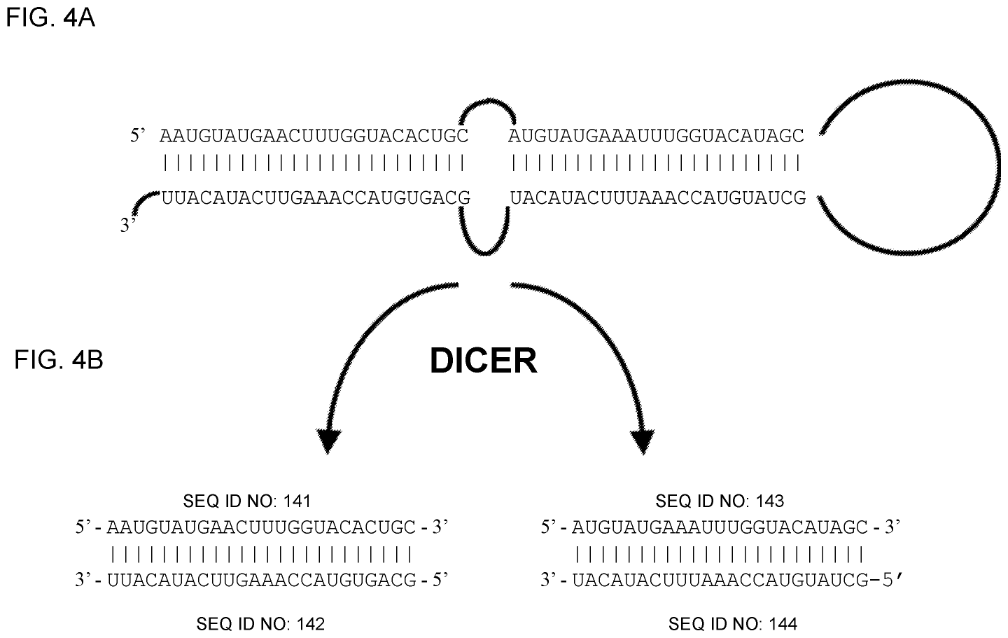

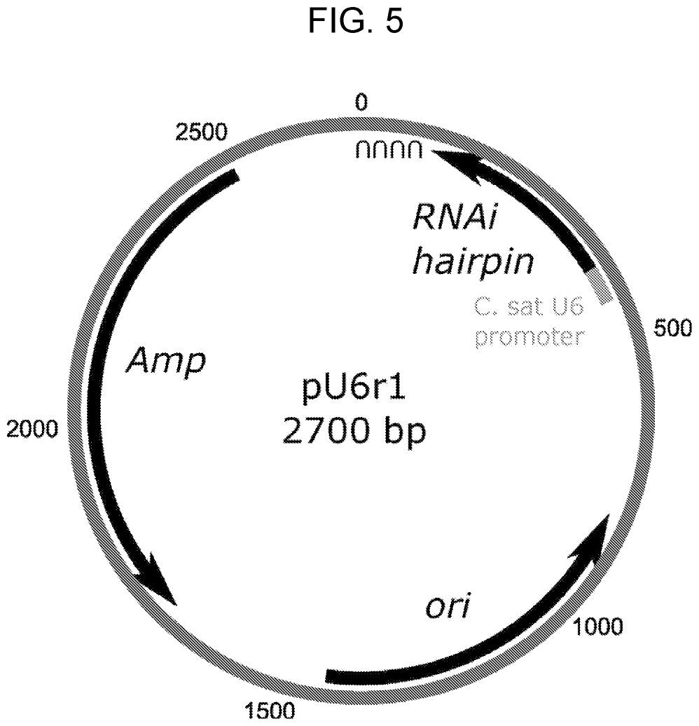

Directly below the analysis of SEQ ID NO: 17 in FIG. 2B, a second nucleotide sequence (SEQ ID NO: 28) is shown that matches the motifs from the C. sativa and C. ruderalis THCAS/CBCAS sequence of SEQ ID NO: 77 and the C. sativa THCAS/CBCAS sequence of SEQ ID NO: 80, and siRNAs transcribed from its complement and reverse complement inhibit THCAS/CBCAS genes that those transcribed from SEQ ID NO: 17 may not. However, the second nucleotide sequence is mismatched with the C. sativa motif in SEQ ID NO: 67 at five positions. Shown below the comparison of SEQ ID NO: 28, three additional nucleotide sequences are compared with the C. sativa motif in SEQ ID NO: 67. Complements and reverse complements of these three nucleotide sequences inhibit THCAS/CBCAS gene targets with the motif comprised in SEQ ID NO: 67, as the first nucleotide sequence (SEQ ID NO: 7) matches the motif in SEQ ID NO: 67, the second (SEQ ID NO: 8) has a single mismatched nucleotide, and the third (SEQ ID NO: 9) contains two mismatches.

Lowermost in FIG. 2B, the sequence of the extremely highly conserved Cannabis CBDAS motif (SEQ ID NO: 101) that corresponds to SEQ ID NO: 1 in Cannabis THCAS/CBCAS is shown. The complement and reverse complement of SEQ ID NO: 101 inhibit C. sativa and C. ruderalis CBDAS gene targets comprising the C. sativa sequence of SEQ ID NO: 112, and the C. ruderalis and C. sativa sequence of SEQ ID NO: 120, both of which comprise this extremely highly conserved motif. In contrast, the comparison of the Cannabis THCAS/CBCAS target sequence of SEQ ID NO: 28 with Cannabis CBDAS shows that the CBDAS motif is mismatched with SEQ ID NO: 28 at nine positions, and therefore siRNAs targeting the Cannabis THCAS/CBCAS genes containing their highly homologous motifs do not inhibit Cannabis CBDAS expression.

FIGS. 3A-3B include an alignment of exemplary nucleotide sequences that form the basis for selective targeting of Cannabis THCAS/CBCAS and CBDAS genes. The nucleotide sequences shown contain the region including the extremely highly conserved motif in Cannabis CBDAS, and the corresponding motif in Cannabis THCAS/CBCAS. The few but extremely highly conserved differences between Cannabis CBDAS and Cannabis THCAS/CBCAS in this motif, together with the presence of Cannabis CBDAS in a single gene copy, allow for efficient and specific inhibition and/or silencing of THCAS/CBCAS. Results from de novo sequencing of THCAS/CBCAS and sequences identified in available database sequences are shown, representing species and varieties (i.e., C. ruderalis, C. sativa, and C. sativa/indica) across the genus.

FIG. 3A includes representative, non-redundant Cannabis THCAS/CBCAS nucleotide sequences from all twelve copies in C. ruderalis, C. sativa, and C. sativa/indica, showing the high homology between these genes in the transcribed segment containing the target motif (SEQ ID NO: 2). Nucleotide positions in the motif that distinguish THCAS/CBCAS from CBDAS are highlighted in black. From this comprehensive sequence list, those in the art are able to immediately recognize the set of siRNA-encoding targeting sequences that target Cannabis THCAS/CBCAS genes with an acceptable number of mismatches over specific length of targeting sequence that comprise distinguishing nucleotides within the motif.

FIG. 3B includes representative, non-redundant Cannabis CBDAS nucleotide sequences from C. ruderalis, C. sativa, and C. sativa/indica. All contain the identical motif of SEQ ID NO: 103. Nucleotides distinguishing CBDAS from characteristic THCAS/CBCAS sequences are highlighted in black. Together with the list of THCAS/CBCAS sequences displayed in FIG. 3A, those in the art are able to immediately recognize the set of siRNA-encoding targeting sequences that specifically inhibit some or all copies of Cannabis THCAS/CBCAS without significantly affecting CBDAS expression (e.g., without resulting in significant inhibition of CBDAS).

FIGS. 4A-4B include an illustration of the mechanism by which some embodiments herein decrease or effectively silence the expression of some or all genomic copies of THCAS and CBCAS without significantly affecting the expression of CBDAS, thereby yielding a THC- and CBC-null Cannabis plant material with a novel chemotype. The structure of an exemplary hpRNA molecule is shown in FIG. 4A, in this example comprising a stem structure with two siRNA regions (left: SEQ ID NO: 141 hybridized to SEQ ID NO: 142; right: SEQ ID NO: 143 hybridized to SEQ ID NO: 144, ribonucleotide sequences set forth in the Sequence Listing without respect to 5′ to 3′ orientation) connected by a linker polyribonucleotide that forms a loop structure in the hpRNA molecule. The specific polyribonucleotide used as the linker may be selected from any of the myriad options known in the art to be suitable for the design of hpRNAs. In the example shown, cleavage of the hpRNA by DICER releases two siRNAs (FIG. 4B), formed by hybridized polyribonucleotides SEQ ID NO: 141 and SEQ ID NO: 142 (left), and SEQ ID NO: 143 and SEQ ID NO: 144 (right).

FIG. 5 is a schematic map of a representative plasmid useful in Cannabis transformation such as is described herein. In addition to an origin of replication (ori) and a selection marker (exemplified by ampicillin; amp), it includes a promoter that can provide expression in Cannabis (exemplified by the C. sativa U6 promoter) that is operably linked to an RNAi hairpin encoding sequence.

### SEQUENCE LISTING

The nucleic acid sequences listed in the accompanying Sequence Listing are shown using standard letter abbreviations for nucleotide bases, as defined in 37 C.F.R. § 1.822. The nucleotide sequences listed define molecules (i.e., polynucleotides and polyribonucleotides) having the nucleotide monomers arranged in the manner described. The nucleotide sequences listed also each define a genus of polynucleotides/polyribonucleotides that comprise the nucleotide monomers arranged in the manner described. In view of the redundancy of the genetic code, it is understood by those in the art that a nucleotide sequence including a coding sequence may at least be modified to substitute nucleotides, often in the third (and sometimes second) position of a codon, without impacting the structure of the encoded polypeptide.

Only one strand of each nucleotide sequence is shown, but the complementary strand is included by any reference to the displayed strand. As the complement and reverse complement of a primary nucleic acid sequence are necessarily disclosed by the primary sequence, the complementary sequence and reverse complementary sequence of a nucleotide sequence are included by any reference to the nucleotide sequence, unless it is explicitly stated to be otherwise (or it is clear to be otherwise from the context in which the sequence appears). Furthermore, as it is understood in the art that the ribonucleotide sequence of an RNA strand is determined by the sequence of the DNA from which it was transcribed (but for the substitution of uracil (U) nucleobases for thymine (T)), an RNA sequence is included by any reference to the DNA sequence encoding it. Embodiments herein employ RNAi technology, by which an mRNA molecule is enzymatically degraded in a cell by hybridizing to a polyribonucleotide with a sufficiently complementary or reverse complementary nucleotide sequence. When Cannabis gene sequences are described herein, it is understood that the sequences are useful for designing appropriate polyribonucleotides (and the polynucleotides that encode them) for use in this process.

A computer readable text file, entitled “2K62523.txt (Sequence Listing.txt)” created on or about Sep. 18, 2021, with a file size of 60 KB, contains the Sequence Listing for this application and is hereby incorporated by reference in its entirety. In the accompanying Sequence Listing:

SEQ ID NO: 1 shows the nucleotide sequence of a highly homologous motif in THCAS and CBCAS genes from a number of cultivars representative of the Cannabis genus, where SEQ ID NOs: 2-29 set forth alternative expressions of this motif and surrounding genomic sequences that were constructed from analysis of available sequences and data returned from de novo sequencing of libraries, from hemp and non-hemp C. sativa (including varieties with desirable chemotypes), C. sativa/indica hybrids, and C. ruderalis.

SEQ ID NOs: 30-32 show the nucleotide sequence of the motif within its larger genomic context, determined by the same methodology as SEQ ID NOs: 1-29.

SEQ ID NOs: 33-89 show nucleotide sequences of the highly homologous THCAS and Cannabis CBCAS motif and surrounding genomic sequences representing sequencing data and database analysis. Representative sequence sources: SEQ ID NO: 84: MG996407.1, KJ469379.1, FINOLA (QKVJ02004887.1_13943_RC_CBCAS, QKVJ02001794.1_69162_RC_CBCAS, QKVJ02001794.1_136712_RC_CBCAS, QKVJ02004488.1_6140_CBCAS, QKVJ02004358.1_21713_CBCAS); SEQ ID NO: 85: MC003345.1, MN422091.1, MT338560.1; SEQ ID NO: 86: [MC003345.1, MN422091.1, MT338560.1); SEQ ID NO: 87: FINOLA (QKVJ02001794.1_9423_RC_CBCAS; QKVJ02004136.1_3042_RC_CBCAS); SEQ ID NO: 88: FINOLA (QKVJ02000019.1_535545_92 THCAS, QKVJ02000019.1_589401_92 THCAS, QKVJ02000019.1_618412_92 THCAS, QKVJ02000019.1_650910_92 THCAS, QKVJ02000019.1_709743_ 92THCAS); SEQ ID NO: 89: [FINOLA (QKVJ02000019.1_535545_92 THCAS, QKVJ02000019.1_589401_92 THCAS, QKVJ02000019.1_618412_92 THCAS, QKVJ02000019.1_650910_92 THCAS, QKVJ02000019.1_709743_92THCAS)].

SEQ ID NO: 90 shows a coding sequence shared by two of the twelve genomic copies of Cannabis THCAS/CBCAS from C. ruderalis (Finola). Representative sequence sources: QKVJ02001794.1_9423_RC_CBCAS, QKVJ02004136.1_3042_RC_CBCAS ORF.

SEQ ID NO: 91 shows a coding sequence shared by two other of the twelve genomic copies of Cannabis THCAS/CBCAS from C. ruderalis (Finola). Representative sequence sources: QKVJ02004887.1_13943_RC_CBCAS, QKVJ02001794.1_69162_RC_CBCAS ORF.

SEQ ID NOs: 92-94 show the coding sequences of three other of the twelve genomic copies of Cannabis THCAS/CBCAS from C. ruderalis (Finola). Representative sequence sources: SEQ ID NO: 92: QKVJ02001794.1_136712_RC_CBCAS ORF; SEQ ID NO: 93: QKVJ02004488.1_6140_CBCAS ORF; SEQ ID NO: 94: QKVJ02004358.1_21713_CBCAS ORF.

SEQ ID NO: 95 show a coding sequence shared by the remaining five genomic copies of Cannabis THCAS/CBCAS from C. ruderalis (Finola). Representative sequence sources: QKVJ02000019.1_535545_92 THCAS, QKVJ02000019.1_589401_92 THCAS, QKVJ02000019.1_618412_92 THCAS, QKVJ02000019.1_650910_92 THCAS, QKVJ02000019.1_709743_92THCAS ORF.

SEQ ID NOs: 96-100 show representative examples of genomic nucleotide sequences comprising the copies of Cannabis THCAS/CBCAS from C. ruderalis (Finola) set forth herein as SEQ ID NOs: 90-94, respectively. Representative sequence sources: SEQ ID NO: 96: QKVJ02001794.1_9423_RC_CBCAS, QKVJ02004136.1_3042_RC_CBCAS DNA; SEQ ID NO: 97: QKVJ02004887.1_13943_RC_CBCAS, QKVJ02001794.1_69162_RC_CBCAS DNA; SEQ ID NO: 98: QKVJ02001794.1_136712_RC_CBCAS DNA; SEQ ID NO: 99: QKVJ02004488.1_6140_CBCAS DNA; SEQ ID NO: 100: QKVJ02004358.1_21713_CBCAS DNA

SEQ ID NO: 101 shows the nucleotide sequence of the highly homologous motif in Cannabis CBDAS, corresponding to that in Cannabis THCAS/CBCAS, identified from the same hemp and non-hemp C. sativa, C. sativa/indica hybrids, and C. ruderalis strains, and by the same methods, as the Cannabis THCAS/CBCAS sequences set forth as SEQ ID NOs: 1-100 herein. SEQ ID NOs: 102-108 set forth alternative expressions of this motif and surrounding genomic sequences that were constructed from the analysis of available sequences and sequencing data.

SEQ ID NOs: 109-122 show nucleotide sequences of the highly homologous Cannabis CBDAS motif and surrounding genomic sequences representing sequencing data and database analysis. Representative sequence sources: SEQ ID NO: 109: KP970868.1, MG996438.1, XM_030624886.1; SEQ ID NO: 110: KP970868.1, MG996438.1, XM_030624886.1; SEQ ID NO: 111: KP970868.1, MG996438.1, XM_030624886.1; SEQ ID NO: 112: KP970868.1, MG996438.1, XM_030624886.1; SEQ ID NO: 117: CM011610.1, KP970857.1, KP970858.1, MG996436.1, MG996439.1, FINOLA; SEQ ID NO: 118: CM011610.1, KP970857.1, KP970858.1, MG996436.1, MG996439.1, FINOLA; SEQ ID NO: 119: CM011610.1, KP970857.1, KP970858.1, MG996436.1, MG996439.1, FINOLA; SEQ ID NO: 120: CM011610.1, KP970857.1, KP970858.1, MG996436.1, MG996439.1, FINOLA; SEQ ID NO: 121: CM011610.1, KP970857.1, KP970858.1, MG996436.1, MG996439.1; SEQ ID NO: 122: AB292682.1.

SEQ ID NO: 123 shows the coding sequence of the single genomic copy of Cannabis CBDAS from C. ruderalis (Finola):

SEQ ID NO: 124 shows the genomic nucleotide sequence comprising the coding sequence of Cannabis CBDAS from C. ruderalis (Finola) set forth herein as SEQ ID NO: 123.

SEQ ID NO: 125 shows an example of a nucleotide sequence encoding an hpRNA molecule selectively targeting Cannabis THCAS/CBCAS genes, where first and second sequences (SEQ ID NO: 126 and SEQ ID NO: 127) forming the stem of the hairpin structure are in bold font and are separated by an intervening sequence that is forced into a loop structure by intramolecular hybridization of the stem nucleotides: MATGTATGAMCTTTGGTACAYWGCAAAAGTAAAGTAATTTAAGCWRTGTACCAAAGKTCAT ACATK.

SEQ ID NOs: 128-138 show further examples of nucleotide sequences encoding an hpRNA molecule selectively targeting Cannabis THCAS/CBCAS genes, where stem sequences can be identified by inspection.

SEQ ID NO: 139 shows a nucleotide sequence of a representative example of a plant promoter.

SEQ ID NO: 140 shows the nucleotide sequence of a DNA construct comprising a representative example of a polynucleotide encoding an hpRNA molecule selectively targeting Cannabis THCAS/CBCAS genes, operably linked to a plant promoter.

SEQ ID NOs: 141-144 show the ribonucleotide sequences used in FIG. 4, as they are visible in the illustration (and not as oriented in the secondary structure of the molecules).

### Select Terms

Backcrossing: Backcrossing methods may be used to introduce an exogenous polynucleotide into plants. The backcrossing technique has been widely used for decades to introduce new traits into plants. Jensen, Ed. Plant Breeding Methodology, John Wiley & Sons, Inc., 1988. In a typical backcross protocol, the original variety of interest (recurrent parent) is crossed to a second variety (non-recurrent parent) that carries a polynucleotide to be transferred. The resulting progeny from this cross are then crossed again to the recurrent parent, and the process is repeated until a plant is obtained wherein essentially all of the desired morphological and physiological characteristics of the recurrent plant are recovered in the converted plant, in addition to the transferred polynucleotide from the non-recurrent parent.

Commodity product: As used herein, the term “cannabis commodity product” refers to commodities (a good used in commerce or exchangeable) produced from genetically modified Cannabis plant materials herein. A commodity product may, for example, be selected the group consisting of food products (for example, baked goods (e.g., cookies), beverages (e.g., coffee and soda), candy, and consumable oils, extracts, and concentrates), inhalable products (e.g., cigarettes and vape oils), concentrates (e.g., for use in vaporizers), creams (e.g., face creams and tattoo creams), extracts, flower, hemp, fiber, oils (e.g., body oils, beard oils, and massage oils), medicaments (e.g., Epidiolex™ (cannabidiol) and Sativex™ (Δ-9-tetrahydrocannibinol and cannabidiol in the EU; nabiximols in the US)), salves, ointments, cosmetics, soaps, lip balms, hair products (e.g., shampoos), bath bombs, bath salts, gels (e.g., topical gels), lotions, roll-on skin products and deodorants, patches (e.g., topical patches and transdermal patches), capsules, tablets, strips (e.g., oral, dissolving strips), fragrances/odorants, (rolling) paper, essential oils, flavorings, antibacterial agents, disinfectants, fungicides, herbicides, insecticides, trypanocides, aerosols, textiles, candles, incense, sunscreen, pigments, infusions, medical implants/devices, carbon sequester and any of the foregoing, formulated for human use or use by animals (e.g., pets).

Essentially derived: In some embodiments, manipulations of plants, seeds, or parts thereof may lead to the creation of essentially derived varieties. As used herein, the term “essentially derived” follows the convention set forth by The International Union for the Protection of New Varieties of Plants (UPOV):
[A] variety shall be deemed to be essentially derived from another variety (“the initial variety”) when
   (i) it is predominantly derived from the initial variety, or from a variety that is itself predominantly derived from the initial variety, while retaining the expression of the essential characteristics that result from the genotype or combination of genotypes of the initial variety;
   (ii) it is clearly distinguishable from the initial variety; and
   (iii) except for the differences which result from the act of derivation, it conforms to the initial variety in the expression of the essential characteristics that result from the genotype or combination of genotypes of the initial variety.
      UPOV, Sixth Meeting with International Organizations, Geneva, Oct. 30, 1992 (document prepared by the Office of the Union).

Exogenous: The term “exogenous,” as applied to polynucleotides and/or polyribonucleotides herein, refers to a polynucleotide or polyribonucleotide in a specific environment or context that is not normally present. For example, if a host cell is transformed with a polynucleotide that does not occur in the untransformed host cell in nature, then that polynucleotide is exogenous to the host cell. Similarly, if a genetically modified host cell expresses a polyribonucleotide that does not occur in the wild-type host cell, the polyribonucleotide is exogenous to the host cell. Exogenous polynucleotides herein also specifically include a polynucleotide that is identical in sequence to a polynucleotide already present in a host cell, but that is located in a different cellular or genomic context than the polynucleotide with the same sequence already present in the host cell. For example, a polynucleotide that is integrated in the genome of the host cell in a different location than a polynucleotide with the same sequence is normally integrated in the genome of the host cell (for example, as a polyribonucleotide comprised in an hpRNA-encoding transcription unit) is exogenous to the host cell. Furthermore, a polynucleotide that is present in a plasmid or vector in the host cell is exogenous to the host cell when a polynucleotide with the same sequence is only normally present in the genome of the host cell.

Expression: As used herein, “expression” of a polynucleotide (for example, a gene or a transgene) refers to the process by which the coded information of a transcriptional unit (including, e.g., gDNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, northern blot, RT-PCR, western blot, or in vitro, in situ, or in vivo protein activity assay(s).

Gene Deletion/Gene Knockout: A “gene deletion” or “gene knockout” refers to rendering a specific gene or family of genes inoperable or inactive, and can be carried out by a number of different genetic techniques. In particular embodiments, a gene deletion reduces or eliminates expression of a polypeptide encoded by the target gene(s). In particular embodiments, the expression of the gene(s) is substantially reduced or eliminated. Substantially reduced means that the expression of the gene(s) is reduced by at least 80%, at least 90%, at least 95%, or at least 98% when compared to an endogenous level of expression of the gene. Expression of gene(s) can be determined by a suitable technique (e.g., by measuring transcript or expressed protein levels). Any suitable technique can be used to generate a gene deletion in a plant, such as a Cannabis plant; specific preferred techniques are provided herein.

Heterologous: The term “heterologous,” as applied to polynucleotides and/or polyribonucleotides herein, means of different origin. For example, if a host cell is transformed with a polynucleotide that does not occur in the untransformed host cell in nature, then that polynucleotide is heterologous (and exogenous) to the host cell. Furthermore, different elements (e.g., promoters, enhancers, coding sequences, and terminators) of an expression cassette may be heterologous to one another and/or to the transformed host. Heterologous polynucleotides herein also specifically include a polynucleotide that is identical in sequence to a polynucleotides already present in a host cell, but that is linked to a different regulatory sequence and/or are present at a different copy number in the host cell.

Inhibition: As used herein, the term “inhibition,” when used to describe an effect on a gene, refers to a measurable decrease in the cellular level of mRNA transcribed from the gene and/or peptide, polypeptide, or protein product of the gene. In some examples, expression of a gene may be inhibited such that expression is substantially or essentially eliminated, and the use of the term “inhibit” herein specifically includes both a reduction in gene expression that leads to a measurable characteristic in the organism, in some examples to “substantially eliminate” or “essentially eliminate” (used interchangeably herein) expression, such that the amount of the gene's activity is undetectable or below a significant amount. In some embodiments herein wherein the expression of THCAS in a Cannabis plant material is substantially or essentially eliminated, the Cannabis plant material contains Δ9-THC in an amount less than 0.3%, which is a significant threshold for regulatory approval in some jurisdictions. “Specific inhibition” refers to the inhibition of a target gene or family of genes without consequently affecting expression of other unrelated genes in the cell wherein the specific inhibition is being accomplished.

Isolated: An “isolated” biological component (such as a polynucleotide, polypeptide, or small molecules (e.g., cannabinoids)) has been substantially separated, produced apart from, or purified away from other biological components in the cell of the organism in which the component originated or was made or naturally occurs (i.e., other chromosomal and extra-chromosomal DNA and RNA, and proteins), while effecting a chemical or functional change in the component (e.g., a nucleic acid may be isolated from a chromosome by breaking chemical bonds connecting the nucleic acid to the remaining DNA in the chromosome; or a chemical compound may be converted to a purified form that is effective or more effective for some use(s) because it is removed from the presence of other components, which may be viewed as contaminants). Polynucleotides and small molecules that have been isolated specifically include nucleic acid molecules and cannabinoids purified by standard purification methods. The term also embraces biological components (such as nucleic acid molecules and cannabinoids) prepared by recombinant expression or production in a host organism or host cell, as well as chemically-synthesized versions, including when they are substantially separated or purified away from other biological components in that product milieu.

Locus: As used herein, the term “locus” refers to a position on the genome that corresponds to a gene, a marker thereof, or a measurable characteristic (e.g., a trait). A locus may be unambiguously defined by an oligonucleotide (e.g., a probe) that specifically hybridizes to a polynucleotide at the locus.

Nucleic acid molecule: As used herein, the term “nucleic acid molecule” refers to a polymeric form of nucleotides, which includes in specific examples both or either of sense and anti-sense strands of RNA, cDNA, genomic DNA, and synthetic forms and mixed polymers of the foregoing. The term includes single- and double-stranded forms of DNA and RNA. A nucleic acid molecule can include either or both naturally occurring and modified nucleotides linked together by naturally occurring and/or non-naturally occurring nucleotide linkages. A nucleotide may be a ribonucleotide, deoxyribonucleotide, or modified form of either. A “polynucleotide” refers to a physical contiguous nucleotide polymer, such as may be comprised in a larger nucleic acid molecule. A nucleic acid molecule is usually at least 10 bases in length, unless otherwise specified. By convention, the nucleotide sequence of a nucleic acid molecule is read from the 5′ to the 3′ end of the molecule. The “complement” of a nucleic acid molecule refers to a polynucleotide having nucleobases that may form base pairs with the nucleobases of the nucleic acid molecule (i.e., A-T/U, and G-C).

Some embodiments include nucleic acids comprising a template DNA that is transcribed into an RNA molecule that comprises a polyribonucleotide that hybridizes to a mRNA molecule. In some examples, the template DNA is the complement of the polynucleotide transcribed into the mRNA molecule, present in the 5′ to 3′ orientation, such that RNA polymerase (which transcribes DNA in the 5′ to 3′ direction) will transcribe the polyribonucleotide from the complement that can hybridize to the mRNA molecule. Unless explicitly stated otherwise, or it is clear to be otherwise from the context, the term “complement” therefore refers to a polynucleotide having nucleobases, from 5′ to 3′, that may form base pairs with the nucleobases of a reference nucleic acid. In some examples, the template DNA is the reverse complement of the polynucleotide transcribed into the mRNA molecule. Thus, unless it is explicitly stated to be otherwise (or it is clear to be otherwise from the context), the “reverse complement” of a polynucleotide refers to the complement in reverse orientation. The foregoing is demonstrated in the following illustration:
ATGATGATG polynucleotide
TACTACTAC “complement” of the polynucleotide
CATCATCAT “reverse complement” of the polynucleotide

As used herein, two polynucleotides are said to exhibit “complete complementarity” when every nucleotide of a polynucleotide read in the 5′ to 3′ direction is complementary to every nucleotide of the other polynucleotide when read in the 5′ to 3′ direction. Similarly, a polynucleotide that is completely reverse complementary to a reference polynucleotide will exhibit a nucleotide sequence where every nucleotide of the polynucleotide read in the 5′ to 3′ direction is complementary to every nucleotide of the reference polynucleotide when read in the 3′ to 5′ direction. These terms and descriptions are recognized in the art and are understood by those of ordinary skill in the art.

Some embodiments of the disclosure include hairpin RNA (hpRNA)-forming RNA molecules. In these hpRNA molecules, both a polyribonucleotide that is substantially identical to the complement or reverse complement of a target ribonucleotide sequence in the target mRNA, and a polyribonucleotide that is substantially the reverse complement thereof, may be found in the same molecule, such that the single-stranded transcribed RNA molecule may “fold over” and hybridize to itself over a region comprising both polyribonucleotides (i.e., in a “stem structure” of the hpRNA).

“Nucleic acid molecules” include all polynucleotides, for example: single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), shRNA (small hairpin RNA), mRNA (messenger RNA), miRNA (micro-RNA), hpRNA (hairpin RNA), tRNA (transfer RNAs, whether charged or discharged with a corresponding acylated amino acid), and cRNA (complementary RNA). The term “deoxyribonucleic acid” (DNA) is inclusive of cDNA, gDNA, and DNA-RNA hybrids. The terms “polynucleotide” and “nucleic acid,” and “fragments” thereof will be understood by those in the art as a term that includes both gDNAs, ribosomal RNAs, transfer RNAs, messenger RNAs, operons, and smaller engineered polynucleotides that encode or may be adapted to encode, peptides, polypeptides, or proteins.

Oligonucleotide: An oligonucleotide is a short nucleic acid polymer (a short nucleic acid molecule). Oligonucleotides may be formed by cleavage of longer nucleic acid segments, or by polymerizing individual nucleotide precursors. Automated synthesizers allow the synthesis of oligonucleotides up to several hundred bases in length. Because oligonucleotides may bind to a complementary nucleic acid, they may be used as probes for detecting DNA or RNA. Oligonucleotides composed of DNA (oligodeoxyribonucleotides) may be used in PCR, a technique for the amplification of DNAs. In PCR, the oligonucleotide is typically referred to as a “primer,” which allows a DNA polymerase to extend the oligonucleotide and replicate the complementary strand. Oligonucleotides may also be used in embodiments herein as a probe, either to detect specific polynucleotides or polyribonucleotides as part of an in vitro process, or to detect polynucleotides or polyribonucleotides in a sample from a plant or plant material.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages; for example, alpha anomeric nucleic acids, etc.). The term “nucleic acid molecule” also includes any topological conformation, including single-stranded, double-stranded, partially duplexed, triplexed, hairpinned, circular, and padlocked conformations.

As used herein with respect to DNA, the term “coding polynucleotide,” “structural polynucleotide,” or “structural nucleic acid molecule” refers to a polynucleotide that is ultimately transcribed into an RNA; for example, when placed under the control of appropriate regulatory elements. The boundaries of a coding polynucleotide are determined by a translation start codon at the 5′-terminus and a translation stop codon at the 3′-terminus. Coding polynucleotides include, but are not limited to, gDNA, cDNA, ESTs, and recombinant polynucleotides. As used herein, “transcribed non-coding polyribonucleotide” refers to segments of mRNA molecules such as 5′UTR, 3′UTR, and intron segments that are not translated into a polypeptide. For example, a transcribed non-coding polyribonucleotide may be a polyribonucleotide that natively exists as an intragenic “spacer” in an RNA molecule.

Operably linked: A first polynucleotide is operably linked with a second polynucleotide when the first polynucleotide is in a functional relationship with the second polynucleotide. When recombinantly produced, operably linked polynucleotides are generally contiguous, and, where necessary to join two coding regions, in the same reading frame (e.g., in a translationally fused ORF). However, polynucleotides need not be contiguous to be operably linked. The term, “operably linked,” when used in reference to a regulatory genetic element and a polynucleotide, means that the regulatory element affects the expression of the linked polynucleotide. “Regulatory elements,” “control elements,” or “regulatory sequences” refer to polynucleotides that influence the timing and level/amount of transcription (or RNA processing or stability) of the operably linked polynucleotide. Regulatory sequences include, for example and without limitation promoters, translation leaders, introns, enhancers, stem-loop structures, repressor binding sequences, termination sequences, and polyadenylation recognition sequences. Particular regulatory elements may be located upstream and/or downstream of a polynucleotide operably linked thereto. Also, particular regulatory elements operably linked to a polynucleotide may be located on the associated complementary strand of a double-stranded nucleic acid molecule.

Plant line: As used herein, a “line” refers to a group of plants that display little genetic variation (e.g., no genetic variation) between individuals for at least one trait. Inbred lines may be created by several generations of self-pollination and selection or, alternatively, by vegetative propagation from a single parent using tissue or cell culture techniques. As used herein, the terms “cultivar,” “variety,” and “type” are synonymous, and these terms refer to a line that is used for commercial production.

Plant material: As used herein, the term “plant material” refers to any processed or unprocessed material derived, in whole or in part, from a plant (e.g., a Cannabis plant). For example and without limitation, a plant material may be a plant, plant part, seed, fruit, leaf, root, flower, plant tissue, callus, plant tissue culture, callus culture, plant explant, plant cell, or plant cell culture. In some usages, the term “plant material” encompasses a viable plant. Embodiments herein also specifically include plant materials excluding whole or viable plants, but including all other plant materials incapable of propagation or regeneration into a viable, reproducible plant. Additional embodiments include plant materials that are capable of propagation or regeneration into a viable, reproducible plant.

Promoter: As used herein, the term “promoter” refers to a region of DNA that may be upstream from the start of transcription, and that may be involved in recognition and binding of RNA polymerase and other proteins to initiate transcription. A promoter may be operably linked to a polynucleotide for expression of the polynucleotide in a cell, or a promoter may be operably linked to a polynucleotide encoding a signal peptide that may be operably linked to a polynucleotide for expression in a cell. A “plant promoter” refers to a promoter capable of initiating transcription in plant cells. Examples of promoters under developmental control include promoters that preferentially initiate transcription in certain tissues, such as leaves, roots, seeds, fibers, xylem vessels, tracheids, trichomes, or sclerenchyma. Such promoters are referred to as “tissue-preferred”. Promoters which initiate transcription only in certain tissues are referred to as “tissue-specific”. A “cell type-specific” promoter primarily drives expression in certain cell types in one or more organs, for example, vascular cells in roots or leaves. An “inducible” promoter may be a promoter which may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters. A “constitutive” promoter is a promoter which may be active under most environmental conditions or in most tissue or cell types. A plant promoter that is functional in a Cannabis cell refers to plant promoters capable of directing transcription in at least one cell type in a Cannabis plant under at least one condition or at least one growth stage. Examples of plant promoters that are functional in a Cannabis plant include promoters isolated from Cannabis genes.

Inducible promoters can be used in some embodiments of the disclosure. See Ward et al., Plant Mol. Biol. 22:361-366, 1993. With an inducible promoter, the rate of transcription increases in response to an inducing agent. Exemplary inducible promoters include, but are not limited to: Promoters from the ACEI system that respond to copper; In2 gene from maize that responds to benzenesulfonamide herbicide safeners; Tet repressor from Tn10; and the inducible promoter from a steroid hormone gene, the transcriptional activity of which may be induced by a glucocorticosteroid hormone (Schena et al., Proc. Natl. Acad. Sci. USA 88:0421, 1991).

Exemplary constitutive promoters include, but are not limited to: Promoters from plant viruses, such as the 35S promoter from Cauliflower Mosaic Virus (CaMV); promoters from rice actin genes; ubiquitin promoters; pEMU; MAS; maize H3 histone promoter; and the ALS promoter, Xba1/NcoI fragment 5′ to the Brassica napus ALS3 structural gene (or a polynucleotide similar to said Xba1/NcoI fragment) (International PCT Publication No. WO96/30530).

Additionally, tissue-specific or tissue-preferred promoter may be utilized in some embodiments of the disclosure. Plants transformed with a nucleic acid molecule comprising a polynucleotide operably linked to a tissue-specific promoter may produce the product of the coding polynucleotide exclusively, or preferentially, in a specific tissue. Exemplary tissue-specific or tissue-preferred promoters include, but are not limited to: a seed-preferred promoter, such as that from the phaseolin gene, a leaf-specific and light-induced promoter, such as that from cab or rubisco, an anther-specific promoter, such as that from LAT52, a pollen-specific promoter such as that from Zm13, and a microspore-preferred promoter, such as that from apg.

Sequence identity: The term “sequence identity” or “identity,” as used herein in the context of two nucleotide sequences, refers to the residues in the two sequences that are the same when aligned for maximum correspondence over a specified comparison window. The term “percentage of sequence identity” may refer to the value determined by comparing two optimally aligned nucleotide sequences over a comparison window, wherein the portion of the nucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity.

Methods for aligning sequences for comparison are well-known in the art. Various programs and alignment algorithms are described in, for example: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-44, 1988; Higgins and Sharp, CABIOS 5:151-3, 1989; Corpet et al., Nucleic Acids Res. 16:10881-90, 1988; Huang et al., Comp. Appl. Biosci. 8:155-65, 1992; Pearson et al., Methods Mol. Biol. 24:307-31, 1994; Tatiana et al., FEMS Microbiol. Lett. 174:247-50, 1999. A detailed consideration of sequence alignment methods and homology calculations can be found in, e.g., Altschul et al., J. Mol. Biol. 215:403-10, 1990.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleotide sequences with even greater similarity to the reference sequences will show increasing percentage identity when assessed by this method.

Specifically hybridizable/Specifically complementary: As used herein, the terms “specifically hybridizable,” “specifically complementary,” and “specifically reverse complementary” indicate a sufficient degree of complementarity or reverse complementarity such that stable and specific binding occurs between a polyribonucleotide and a nucleic acid molecule comprising a target polyribonucleotide. As is well-known in the art, a polyribonucleotide need not be 100% complementary to its target polyribonucleotide to be specifically hybridizable. In RNAi applications using hpRNAs, the lower free energy required for intramolecular hybridization (as compared to intermolecular hybridization) facilitates the hybridization of partially complementary or reverse complementary primary transcripts (for example, transcripts comprising loop-forming sequences and non-hybridizing sequences in a stem between siRNA sequences).

As used herein, the term “identical,” “substantial identity,” “substantially homologous,” or “substantial homology,” with regard to a reference polyribonucleotide, refers to a polyribonucleotide having contiguous nucleotides that hybridize to a polyribonucleotide or oligonucleotide consisting of the nucleotide sequence of the reference polyribonucleotide. For example, an siRNA consisting of the polyribonucleotide encoded by any of SEQ ID NOs: 17, 19, 24, 28, 43, and 80 is substantially homologous or substantially identical to a reference polyribonucleotide if the siRNA hybridizes to the reference polyribonucleotide. Substantially identical polyribonucleotides herein (e.g., siRNAs) share at least 80% sequence identity.

In examples herein, substantially identical polyribonucleotides have between 80% and 100% sequence identity. In particular examples, substantially identical polyribonucleotides have between 80% and 100% sequence identity. In other examples, substantially identical polyribonucleotides have between 85% and 100% sequence identity. In other examples, substantially identical polyribonucleotides have between 86% and 100% sequence identity. In other examples, substantially identical polyribonucleotides have between 87% and 100% sequence identity. In other examples, substantially identical polyribonucleotides have between 88% and 100% sequence identity. In other examples, substantially identical polyribonucleotides have between 89% and 100% sequence identity. In other examples, substantially identical polyribonucleotides have between 90% and 100% sequence identity. In other examples, substantially identical polyribonucleotides have between 91% and 100% sequence identity. In other examples, substantially identical polyribonucleotides have between 92% and 100% sequence identity. In other examples, substantially identical polyribonucleotides have between 93% and 100% sequence identity. In other examples, substantially identical polyribonucleotides have between 94% and 100% sequence identity. In other examples, substantially identical polyribonucleotides have between 95% and 100% sequence identity. In yet other examples, substantially identical polyribonucleotides have between 96% and 100% sequence identity.

The property of substantial identity is closely related to specific hybridization. For example, a polyribonucleotide is specifically hybridizable when there is a sufficient degree of complementarity or reverse complementarity to avoid non-specific binding of the polyribonucleotide to non-target polyribonucleotides. In specific embodiments herein, an RNA molecule (e.g., hpRNA and siRNA molecules) comprises a polyribonucleotide that is specifically hybridizable to a Cannabis THCAS/CBCAS sequence that comprises the highly homologous cannabinoid synthase motif described herein over at least 12 (e.g., at least 15) contiguous nucleotides of the motif.

Stability: As used herein, the term “stability,” or “stable,” refers to a given plant component or trait that is heritable and is maintained at substantially the same level through multiple seed generations, under same or similar conditions. For example, a stable component or trait may be maintained for at least three generations at substantially the same level. In this context, the term “substantially the same” refers in some embodiments to a component maintained to within 25% between two different generations; within 20%; within 15%; within 10%; within 5%; within 3%; within 2%; and/or within 1% between two different generations, as well as a component that is maintained perfectly between two different generations. In some embodiments, a stable plant component may be selected from among, for example and without limitation, oil components, fiber components, and cannabinoid components. The stability of a component may be affected by one or more environment factors. For example, the stability of an oil component may be affected by, for example and without limitation, temperature, location, stress, and the time of planting. Subsequent generations of a plant having a stable component under (the same or similar) field conditions will be expected to produce the plant component in a similar manner, for example, as set forth above.

Trait or phenotype: The terms “trait” and “phenotype” are used interchangeably herein. As used herein, “phenotype” refers to a stabilized, heritable characteristic of a plant, manifesting under specific environmental conditions, such as may be detected upon inspection or examination during one or more specific stages of growth, or by analysis (e.g., chemical analysis) of the plant or tissues, materials, or products derived from or used to produce the plant. For the purposes of the present disclosure, phenotypes specifically include chemotypes; for example, characteristic abundance or relative amounts of cannabinoids or other Cannabis-derived chemicals. In some embodiments, phenotypes include agronomic traits affecting growth and/or yield of the plant.

Transformation: As used herein, the term “transformation” refers to the transfer of one or more polynucleotide(s) into a cell. A cell is “transformed” by or with a polynucleotide when a nucleic acid molecule comprising the polynucleotide is introduced into the cell, and the polynucleotide becomes stably replicated by the cell, either by incorporation of the nucleic acid molecule into the cellular genome, or by episomal replication. As used herein, the term “transformation” encompasses all techniques by which a nucleic acid molecule can be introduced into such a cell. Examples include, but are not limited to: transfection with viral vectors; transformation with plasmid vectors; electroporation (Fromm et al., Nature 319:791-3, 1986); lipofection (Feigner et al., Proc. Natl. Acad. Sci. USA 84:7413-7, 1987); microinjection (Mueller et al., Cell 15:579-85, 1978); Agrobacterium-mediated transfer (Fraley et al., Proc. Natl. Acad. Sci. USA 80:4803-7, 1983); direct DNA uptake; and microprojectile bombardment (Klein et al., Nature 327:70, 1987).

Transgene: The term “transgene” refers to an exogenous polynucleotide in the genome of an organism. In some examples, a transgene may be a DNA that encodes an hpRNA molecule that comprises a polyribonucleotide that hybridizes to an endogenous mRNA molecule found in Cannabis. In these and other examples, a transgene may contain regulatory elements operably linked to a polynucleotide of the transgene (e.g., a promoter).

Variety or cultivar: The terms “variety” or “cultivar” refer herein to a plant line that is used for commercial production and/or for research, which plant line is distinct, stable and uniform in its characteristics when propagated. In the case of a hybrid variety or cultivar, the parental lines are distinct, stable, and uniform in their characteristics. The term “Cannabis,” as used herein, includes all species of the Cannabis genus, specifically including fiber-type or hemp-type C. sativa, C. indica, or C. ruderalis, varieties of Cannabis species characterized as “high-CBD,” and high-intoxicant or high-THC C. sativa or C. indica varieties.

Vector: “Vectors” include nucleic acid molecules as introduced into a cell, for example, to produce a transformed cell. A vector may include genetic elements that permit it to replicate in the host cell, such as an origin of replication. Examples of vectors include, but are not limited to: a plasmid; cosmid; bacteriophage; or virus that carries exogenous DNA into a cell. A vector may include one or more polynucleotide, including those that encode hpRNA molecules, and/or selectable marker genes and/or other genetic elements known in the art. A vector may transduce, transform, or infect a cell, thereby causing the cell to express RNA molecules and/or proteins encoded by the vector. A vector optionally includes materials to aid in achieving entry of the nucleic acid molecule into the cell (e.g., a liposome, protein coating, etc.). FIG. 5 provides a graphic representation of an exemplar vector.

Unless specifically indicated or implied, the terms “a,” “an,” and “the” signify “at least one,” as used herein.

Unless otherwise specifically explained, all technical and scientific terms used herein have the same meaning as commonly understood by those of ordinary skill in the art to which this disclosure belongs. Definitions of common terms in molecular biology can be found in, for example, Lewin's Genes X, Jones & Bartlett Publishers, 2009 (ISBN 10 0763766321); Krebs et al. (eds.), The Encyclopedia of Molecular Biology, Blackwell Science Ltd., 1994 (ISBN 0-632-02182-9); and Meyers R. A. (ed.), Molecular Biology and Biotechnology: A Comprehensive Desk Reference, VCH Publishers, Inc., 1995 (ISBN 1-56081-569-8). All percentages are by weight and all solvent mixture proportions are by volume unless otherwise noted. All temperatures are in degrees Celsius.

### DETAILED DESCRIPTION

### 1. Overview of Several Embodiments

Cannabinoids are a valuable class of chemical compounds that may be useful for the treatment and/or prevention of chronic pain, neuropathic pain, anorexia, nausea, cancer/AIDS wasting, spasticity, and immunodeficiency. Embodiments of the present disclosure address a problem in the art. Five copies of THCAS, seven copies of CBCAS, and a single copy of CBDAS are distributed in three distinct chromosomal regions in Cannabis. Attempting to target particular cannabinoid synthase sequences by RNAi (for example, to produce a THC-null Cannabis plant) is either unsuccessful, or else results in an unstable phenotype, due to recombination between the multiple copies of highly related cannabinoid synthase genes in the cannabis genome.

Embodiments herein address this problem by targeting a unique sequence motif that is conserved in and between particular cannabinoid synthases, thereby modifying the cannabinoid biosynthetic pathway to alter (e.g., increase) the production of cannabinoids (in embodiments, specifically to increase cannabinoids other than THC) that are typically found in lesser amounts in wild-type plants and existing varieties. It is recognized that alteration in one cannabinoid may result in concomitant alteration(s) in other cannabinoids, for instance due to the shunting of production from one to another. For instance, by decreasing the production of THC, other cannabinoid(s) are produced at a higher level; the inverse is also true.

In particular embodiments herein, hpRNAs comprising polyribonucleotides targeting this unique sequence motif in THCAS/CBCAS (e.g., SEQ ID NOs: 17, 19, 24, 28, 43, and 80) while avoiding off-target inhibition of CBDAS are utilized to decrease or substantially eliminate expression of every THCAS/CBCAS gene in the cannabis genome. In additional examples, CRISPR is used to target a shared sequence motif as described herein, in order to eliminate (or substantially eliminate) expression of every THCAS/CBCAS gene in the cannabis genome. Plants produced by these strategies both provide novel chemotypes, and constitute a THC-null and CBC-null engineering platform that may be used to introduce further desirable novel chemotypes, for example, by introducing or further modifying other biosynthetic machinery in the plant.

In other embodiments provided herein, hpRNAs comprising polyribonucleotides targeting a herein described unique sequence motif in CBDAS (SEQ ID NO: 103) while avoiding off-target inhibition of THCAS/CBCAS are utilized to decrease or substantially eliminate expression of the CBDAS gene in the Cannabis genome. In additional examples, embodiments involve using CRISPR to target a unique sequence motif (such as that shown in SEQ ID NO: 103) as described herein, in order to eliminate (or substantially eliminate) expression of the CBDAS gene in the cannabis genome. Plants produced by this strategy provide novel chemotypes, and constitute an enhanced-THC/CBC engineering platform that may be used to introduce further desirable novel chemotypes, for example, by introducing or further modifying other biosynthetic machinery in the plant.

Also provided are alternative methods for making the modifications to THCAS/CBCAS or CBDAS.

### II. THCAS- and CBCAS-Inhibitory Nucleic Acid Molecules

Described herein are nucleic acid molecules useful for decreasing the expression of THCAS and CBCAS genes in Cannabis.

Some embodiments herein include polynucleotides engineered to encode hpRNA molecules comprising polyribonucleotides that target the highly-conserved motif in THCAS and CBCAS genes defined by SEQ ID NO: 1 and SEQ ID NOs: 2-29, and that are effective to decrease the expression of multiple copies (e.g., every copy) of THCAS and CBCAS in the cannabis genome. Particular embodiments include polynucleotides that encode hpRNA molecules comprising a polyribonucleotide that targets the highly-conserved motif of SEQ ID NOs: 17, 19, 24, 28, 43, and 80. These and further embodiments include the encoded hpRNA molecules, which are processed in the cell into siRNA, miRNA, and/or shRNA molecules that specifically hybridize to THCAS and CBCAS mRNAs in Cannabis plants comprising the target polynucleotides. Nucleic acid molecules described herein, when introduced into a Cannabis plant or plant material, initiate RNAi in the plant or plant material, consequently reducing, significantly reducing, substantially eliminating, or eliminating expression of the THCAS and/or CBCAS. In some examples, reduction or elimination of the expression of the THCAS and/or CBCAS results in a modified chemotype in the host plant.

In the cell, hpRNA molecules are modified through a ubiquitous enzymatic process to generate siRNA molecules. This enzymatic process may utilize an RNase III enzyme, such as DICER in eukaryotes, either in vitro or in vivo. See Elbashir et al., Nature 411:494-8, 2001; and Hamilton and Baulcombe, Science 286(5441):950-2, 1999. DICER or functionally-equivalent RNase III enzymes cleave larger dsRNA strands and/or hpRNA molecules into smaller oligonucleotides, siRNAs and miRNAs. The siRNA molecules produced by these enzymes have 2 to 3 nucleotide 3′ overhangs, and 5′ phosphate and 3′ hydroxyl termini. The siRNA molecules generated by RNase III enzymes are unwound and separated into single-stranded RNA in the cell. The siRNA molecules then specifically hybridize with mRNAs transcribed from the target gene, and both RNA molecules are subsequently degraded by an inherent cellular RNA-degrading mechanism. This process results in the effective degradation or removal of the mRNA encoded by the target gene in the target organism. The outcome is the post-transcriptional silencing of the targeted gene. In embodiments herein, siRNA molecules produced by endogenous RNase III enzymes from the hpRNAs of the disclosure efficiently mediate the inhibition of THCAS and/or CBCAS in Cannabis.

It is a feature of the disclosure that the RNAi post-transcriptional inhibition system tolerates sequence variations among target genes that might be expected due to genetic mutation, strain polymorphism, or evolutionary divergence. The antisense polyribonucleotide does not need to be absolutely identical to the complement or reverse complement of either a primary transcription product or a fully-processed mRNA of the target gene thereof, so long as the antisense polyribonucleotide is specifically hybridizable to either a primary transcription product or a fully-processed mRNA of the target gene

Taking into account the tolerance of the RNAi mechanism for slight sequence variations, inhibition of THCAS/CBCAS using the hpRNAs of the present disclosure is sequence-specific; i.e., antisense polyribonucleotides are utilized for specific inhibition of THCAS and/or CBCAS (for example, for specific inhibition of THCAS and CBCAS) without significantly affecting expression of the CBDAS gene.

Accordingly, the antisense polyribonucleotide utilized for specific inhibition of THCAS/CBCAS in some embodiments comprises at least 12 contiguous nucleotides of the complement or reverse complement of the motif defined by SEQ ID NO: 1 and SEQ ID NOs: 2-29; for example, the motif of any of SEQ ID NOs: 17, 19, 24, 28, 43, and 80. In particular embodiments, the antisense polyribonucleotide comprises at least 12 contiguous nucleotides of the complement or reverse complement of the motif defined by SEQ ID NO: 17 or SEQ ID NO: 28. The antisense polyribonucleotide comprising at least 12 contiguous nucleotides of the complement or reverse complement of the motif comprises one of at least 12, at least 13, at least 14, at least 15, at least 16, at least 17, at least 18, at least 19, at least 20, at least 21, at least 22, at least 23, or 24 contiguous nucleotides of the complement or reverse complement of the motif in particular examples. This specifically means that the antisense polyribonucleotide may comprise 12, 13, 14, 15, 16, 17, 18, 19, 20, 21, 22, 23, or 24 contiguous nucleotides of the complement or reverse complement of the motif.

In alternative embodiments, the antisense polyribonucleotide utilized for specific inhibition of THCAS/CBCAS comprises a nucleotide sequence that is at least 90% identical to the complement or reverse complement of the motif defined by SEQ ID NO: 1 and SEQ ID NOs: 2-29; for example, the motif of any of SEQ ID NOs: 17, 19, 24, 28, 43, and 80. In some examples of these embodiments, the antisense polyribonucleotide comprises a nucleotide sequence that is at least 90% identical to the complement or reverse complement of the motif defined by SEQ ID NO: 17 or SEQ ID NO: 28. The antisense polyribonucleotide comprising a nucleotide sequence that is at least 90% identical to the complement or reverse complement of the motif is at least 90%, at least 91%, at least 92%, at least 93%, at least 94%, at least 95%, at least 96%, at least 97%, at least 98%, at least 99%, or 100% identical to the complement or reverse complement of the motif in particular examples. This specifically means that the antisense polyribonucleotide may be 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% identical to the complement or reverse complement of the motif.

In any of the embodiments described above, the antisense polyribonucleotide utilized for specific inhibition of THCAS and/or CBCAS may be engineered such that it does not comprise 5 or more (for example, 8 or more) contiguous nucleotides of the complement or reverse complement of the motif defined by SEQ ID NO: 103. In any of the embodiments described above, the antisense polyribonucleotide may also or alternatively be engineered such that it is less than 92% identical to the complement or reverse complement of the motif defined by SEQ ID NO: 103. This specifically means that the antisense polyribonucleotide may comprise less than 21, less than 20, less than 19, less than 18, less than 17, less than 16, less than 15, less than 14, less than 13, less than 12, less than 11, less than 10, less than 9, less than 8, less than 7, less than 6, or less than 5 contiguous nucleotides of the complement or reverse complement of the motif. This also specifically means that the antisense polyribonucleotide may be less than 91.5%, less than 91%, less than 90.5%, less than 90%, less than 89.5%, or less than 80%, less than 79.5%, less than 79%, less than 78.5%, less than 78%, less than 77.5%, less than 77%, less than 76.5%, less than 76%, less than 75.5%, or less than 75% identical to the complement or reverse complement of the motif.

An hpRNA molecule herein may be transcribed from a polynucleotide containing an antisense nucleotide sequence that encodes any of the foregoing antisense polyribonucleotides; a sense nucleotide sequence that is substantially identical or identical to the antisense polyribonucleotide; and an intervening polyribonucleotide positioned between the sense and the antisense polyribonucleotides, such that the sense and antisense polyribonucleotides in the transcript of the polynucleotide hybridize to form all or part of a “stem” structure in the hpRNA molecule, and the polyribonucleotide transcribed from the intervening sequence forms a “loop.” For ease of explanation, these polyribonucleotides may be listed in the order in which they appear in the 5′ to 3′ direction in the hpRNA: the first polyribonucleotide, a second polyribonucleotide (the intervening, spacer polyribonucleotide), and a third polyribonucleotide (substantially identical or identical to the complement or reverse complement of the first polyribonucleotide). In some embodiments, the hpRNA molecule comprises a plurality of such sense and corresponding antisense polyribonucleotides present in the stem of the hpRNA, which may be, for example, separated by intervening sequences in each strand of the stem (see FIG. 4A). In some embodiments, the sense and corresponding antisense polyribonucleotides have different lengths.

The intervening, spacer polyribonucleotide may comprise any suitable sequence that facilitates secondary structure formation between the polyribonucleotides of the stem structure. In some examples, the spacer is part of a sense or antisense polyribonucleotide in the hpRNA. In further examples, the spacer is an intron. In some embodiments, however, the hpRNA does not comprise a spacer. Many suitable spacers are known and widely-used in the art to engineer hpRNAs that are processed into siRNAs in planta, and any of the spacers may be used in embodiments herein, according to the discretion of the practitioner.

In some embodiments herein, the hpRNA molecule comprises at least two antisense polyribonucleotides and the same number of substantially complementary or reverse complementary sense polyribonucleotides arranged in the hpRNA molecule to be in position to hybridize in a stem structure, wherein each of the antisense polyribonucleotides in the hpRNA targets the highly-conserved motif (for example, SEQ ID NO: 17 and/or SEQ ID NO: 28) in a THCAS/CBCAS gene. In particular embodiments, one of the antisense polyribonucleotides in the hpRNA targets the highly-conserved motif in a gene annotated or specifically identified in a particular Cannabis plant as a THCAS gene (e.g., SEQ ID NO: 17), and another of the antisense polyribonucleotides targets the highly-conserved motif in a gene annotated or specifically identified in the Cannabis plant as a CBCAS gene (e.g., SEQ ID NO: 28). Therefore, some examples herein specifically include a polynucleotide comprising two nucleotide sequences encoding corresponding sense and antisense polyribonucleotides, and two nucleotide sequences encoding specifically complementary or reverse complementary polyribonucleotides positioned on the other single strand of the hpRNA molecule. In specific examples, the polynucleotide comprises more than 2 nucleotide sequences encoding corresponding sense and antisense polyribonucleotides. In such examples, the polynucleotide may specifically comprise 3 nucleotide sequences encoding corresponding sense and antisense polyribonucleotides.

In particular embodiments, an antisense polyribonucleotide of an hpRNA molecule is formed by transcription from a polynucleotide comprising a nucleotide sequence that is substantially identical to the complement or reverse complement of any of SEQ ID NOs: 33-89, wherein the nucleotide sequence comprises at least 12 contiguous nucleotides of the complement or reverse complement of the motif defined by SEQ ID NO: 1 and SEQ ID NOs: 2-29 (for example, the motif of any of SEQ ID NOs: 17, 19, 24, 28, 43, and 80). In these and other particular embodiments, the antisense polyribonucleotide of an hpRNA molecule is formed by transcription from a polynucleotide comprising a nucleotide sequence that is at least 90% identical to the complement or reverse complement of the motif defined by SEQ ID NO: 1 and SEQ ID NOs: 2-29; for example, the motif of any of SEQ ID NOs: 17, 19, 24, 28, 43, and 80.

In particular embodiments, a sense polyribonucleotide of an hpRNA molecule may be formed by transcription from a polynucleotide comprising a nucleotide sequence that is substantially identical to any of SEQ ID NOs: 33-89, wherein the nucleotide sequence comprises at least 12 contiguous nucleotides of the motif defined by SEQ ID NO: 1 and SEQ ID NOs: 2-29 (for example, the motif of any of SEQ ID NOs: 17, 19, 24, 28, 43, and 80). In these and other particular embodiments, the sense polyribonucleotide of an hpRNA molecule is formed by transcription from a polynucleotide comprising a nucleotide sequence that is at least 90% identical to the motif defined by SEQ ID NO: 1 and SEQ ID NOs: 2-29; for example, the motif of any of SEQ ID NOs: 17, 19, 24, 28, 43, and 80.

In some embodiments, an hpRNA molecule may be formed by transcription from a polynucleotide selected from the group consisting of SEQ ID NOs: 128-138; for example, a polynucleotide selected from the group consisting of SEQ ID NOs: 131-138. In particular examples, the polynucleotide is SEQ ID NO: 137.

Some embodiments of the disclosure include introduction of a recombinant nucleic acid molecule of the present disclosure into a Cannabis plant (i.e., transformation) to achieve THCAS-inhibitory and/or CBCAS-inhibitory levels of expression of hpRNA molecules. Such recombinant DNA molecule may, for example, be a vector, such as a linear or a closed circular plasmid. The vector system may be a single vector or plasmid, or two or more vectors or plasmids that together contain the total DNA to be introduced into the genome of a host. In addition, a vector may be an expression vector. Polynucleotides of the disclosure can, for example, be suitably inserted into a vector under the control of a suitable promoter that functions in Cannabis to drive expression of the linked polynucleotide. Many suitable vector systems are available for the purpose of transforming plants, and selection of the appropriate vector system used to introduce the polynucleotide into the Cannabis plant material is within the practitioner's discretion.

Promoters suitable for use in nucleic acid molecules of the disclosure include those that are inducible, viral, synthetic, or constitutive, all of which are well known in the art. Non-limiting examples describing such promoters include U.S. Pat. No. 6,437,217 (maize RS81 promoter); U.S. Pat. No. 5,641,876 (rice actin promoter); U.S. Pat. No. 6,426,446 (maize RS324 promoter); U.S. Pat. No. 6,429,362 (maize PR-1 promoter); U.S. Pat. No. 6,232,526 (maize A3 promoter); U.S. Pat. No. 6,177,611 (constitutive maize promoters); U.S. Pat. Nos. 5,322,938, 5,352,605, 5,359,142, and U.S. Pat. No. 5,530,196 (CaMV 35S promoter); U.S. Pat. No. 6,433,252 (maize L3 oleosin promoter); U.S. Pat. No. 6,429,357 (rice actin 2 promoter, and rice actin 2 intron); U.S. Pat. No. 6,294,714 (light-inducible promoters); U.S. Pat. No. 6,140,078 (salt-inducible promoters); U.S. Pat. No. 6,252,138 (pathogen-inducible promoters); U.S. Pat. No. 6,175,060 (phosphorous deficiency-inducible promoters); U.S. Pat. No. 6,388,170 (bidirectional promoters); U.S. Pat. No. 6,635,806 (gamma-coixin promoter); and U.S. Patent Publication No. 2009/757,089 (maize chloroplast aldolase promoter). Additional promoters include the nopaline synthase (NOS) promoter (Ebert et al., Proc. Natl. Acad. Sci. USA 84(16):5745-9, 1987) and the octopine synthase (OCS) promoters (which are carried on tumor-inducing plasmids of Agrobacterium tumefaciens); the caulimovirus promoters such as the cauliflower mosaic virus (CaMV) 19S promoter (Lawton et al., Plant Mol. Biol. 9:315-24, 1987); the CaMV 35S promoter (Odell et al., Nature 313:810-2, 1985; the figwort mosaic virus 35S-promoter (Walker et al., Proc. Natl. Acad. Sci. USA 84(19):6624-8, 1987); the sucrose synthase promoter (Yang and Russell, Proc. Natl. Acad. Sci. USA 87:4144-8, 1990); the R gene complex promoter (Chandler et al., Plant Cell 1:1175-83, 1989); the chlorophyll a/b binding protein gene promoter; CaMV 35S (U.S. Pat. Nos. 5,322,938, 5,352,605, 5,359,142, and 5,530,196); FMV 35S (U.S. Pat. Nos. 6,051,753, and 5,378,619); a PC1SV promoter (U.S. Pat. No. 5,850,019); the SCP1 promoter (U.S. Pat. No. 6,677,503); and AGRtu.nos promoters (GenBank™ Accession No. V00087; Depicker et al., J. Mol. Appl. Genet. 1:561-73, 1982; Bevan et al., Nature 304:184-7, 1983). In particular embodiments, nucleic acid molecules of the disclosure comprise a tissue-specific promoter, such as a trichome-specific or flower-specific promoter, operably linked to the hpRNA-encoding polynucleotide. An example a promoter that may be used to drive expression in embodiments herein is SEQ ID NO: 139, a Cannabis sativa homolog of the U6 promoter from Arabidopsis thaliana. An exemplary vector that includes a U6 promoter is shown in FIG. 5.

Additional regulatory elements that may optionally be operably linked to a polynucleotide in embodiments herein include 5′UTRs located between a promoter and a coding polynucleotide, and 3′ transcription termination regions. These genetic elements may provide regulatory signals capable of affecting transcription or mRNA processing. The polyadenylation signal functions in plants to cause the addition of polyadenylate nucleotides to the 3′ end of the mRNA precursor. The polyadenylation element can be derived from a variety of plant genes, or from T-DNA (transfer DNA, such as from agrobacterium; see e.g., Rommens et al., Plant Physiol. 139(3)1338-1349, 2005) genes. A non-limiting example of a 3′ transcription termination region is the nopaline synthase 3′ region (nos 3′; Fraley et al., Proc. Natl. Acad. Sci. USA 80:4803-7, 1983). An example of the use of different 3′ non-translated regions is provided in Ingelbrecht et al., Plant Cell 1:671-80, 1989. Non-limiting examples of polyadenylation signals include one from a Pisum sativum RbcS2 gene (Ps.RbcS2-E9; Coruzzi et al., EMBO J. 3:1671-9, 1984) and AGRtu.nos (GenBank™ Accession No. E01312).

A recombinant nucleic acid molecule or vector may comprise a selectable marker that confers a selectable phenotype on a transformed Cannabis cell. Selectable markers may also be used to select for plants or plant cells that comprise a recombinant nucleic acid molecule of the disclosure. The marker may encode biocide resistance, antibiotic resistance (e.g., kanamycin, Geneticin (G418), bleomycin, hygromycin, etc.), or herbicide tolerance (e.g., glyphosate, etc.). Examples of selectable markers include, but are not limited to: a neo gene which codes for kanamycin resistance and can be selected for using kanamycin, G418, etc.; a bar gene which codes for bialaphos resistance; a mutant EPSP synthase gene which encodes glyphosate tolerance; a nitrilase gene which confers resistance to bromoxynil; a mutant acetolactate synthase (ALS) gene which confers imidazolinone or sulfonylurea tolerance; and a methotrexate resistant DHFR gene. Multiple selectable markers are available that confer resistance to ampicillin, bleomycin, chloramphenicol, gentamycin, hygromycin, kanamycin, lincomycin, methotrexate, phosphinothricin, puromycin, spectinomycin, rifampicin, streptomycin and tetracycline, and the like. Examples of such selectable markers are illustrated in, for example, U.S. Pat. Nos. 5,550,318; 5,633,435; 5,780,708 and 6,118,047.

A recombinant nucleic acid molecule or vector of the present disclosure may also include a screenable marker. Screenable markers may be used to monitor expression. Exemplary screenable markers include a β-glucuronidase or uidA gene (GUS) which encodes an enzyme for which various chromogenic substrates are known (Jefferson et al., Plant Mol. Biol. Rep. 5:387-405, 1987); an R-locus gene, which encodes a product that regulates the production of anthocyanin pigments (red color) in plant tissues (Dellaporta et al., “Molecular cloning of the maize R-nj allele by transposon tagging with Ac.” In 18th Stadler Genetics Symposium, P. Gustafson and R. Appels, eds. (New York: Plenum), pp. 263-82, 1988); a β-lactamase gene (Sutcliffe et al., Proc. Natl. Acad. Sci. USA 75:3737-41, 1978); a gene which encodes an enzyme for which various chromogenic substrates are known (e.g., PADAC, a chromogenic cephalosporin); a luciferase gene (Ow et al., Science 234:856-9, 1986); an xylE gene that encodes a catechol dioxygenase that can convert chromogenic catechols (Zukowski et al., Gene 46(2-3):247-55, 1983); an amylase gene (Ikatu et al., Bio/Technol. 8:241-2, 1990); a tyrosinase gene which encodes an enzyme capable of oxidizing tyrosine to DOPA and dopaquinone which in turn condenses to melanin (Katz et al., J. Gen. Microbiol. 129:2703-14, 1983); and an α-galactosidase.

### III. Additional Methods of Targeted Genetic Engineering

Additional targeted genetic engineering approaches may be utilized to obtain specific inhibition of every THCAS and CBCAS gene in the Cannabis genome.

In particular embodiments, gene deletion in Cannabis is mediated by a gene editing system such as Clustered Regularly Interspaced Short Palindromic Repeats/CRISPR-associated (CRISPR/Cas), transcription activator-like effector nucleases (TALENs), zinc-finger nucleases (ZFNs), and/or meganucleases. In particular embodiments, a gene or set of genes (such as all of the THCAS and CBCAS genes) is deleted by targeting a consensus sequence motif in THCAS/CBDAS) (e.g., e.g., SEQ ID NOs: 17, 19, 24, 28, 43, and 80). In particular embodiments, a gene is deleted by introducing one or more mutations that disable the function of a protein encoded by the gene(s). In particular embodiments, a gene is partially or completely removed from the genome of Cannabis.

The widely popular CRISPR/Cas9 system is one method by which targeted disruption is performed. Use of this system in plants is described in Jaganathan et al. (Front Plant Sci. 2018; doi.org/10.3389/fpls.2018.00985), Zhang et al. (Nature Plants, 5:779-794, 2019), Wada et al. (BMC Plant Biology 20, Art. #234, 2020; doi.org/10.1186/s12870-020-02385-5), Mao et al. (NSR 6(3):421-437, 2019; doi.org/10.1093/nsr/nwz005), and broadly described online at en.wikipedia.org/wiki/CRISPR. Use of CRISPR to modify Cannabis has been described in Zhang et al. (Plant Biotech J., pg 1-9, 2021; doi.org/10.1111/pbi.13611).

The CRISPR nuclease system is a prokaryotic immune system that confers resistance to foreign genetic elements such as plasmids and phages and provides a form of acquired immunity. CRISPRs are DNA loci containing short repetitions of base sequences. In the context of a prokaryotic immune system, each repetition is followed by short segments of spacer DNA belonging to foreign genetic elements that the prokaryote was exposed to. This CRISPR array of repeats interspersed with spacers can be transcribed into RNA. The RNA can be processed to a mature form and associate with a Cas (CRISPR-associated) nuclease. A CRISPR-Cas system including an RNA having a sequence that can hybridize to the foreign genetic elements and Cas nuclease can then recognize and cut these exogenous genetic elements in the genome.

A single Cas enzyme can be programmed by a gRNA molecule to site-specifically cleave a specific target nucleic acid. Cas9 is an exemplary Type II CRISPR Cas protein. Cas9 includes two distinct endonuclease domains (HNH and RuvC/RNase H-like domains), one for each strand of the target nucleic acid. RuvC and HNH together produce double-stranded breaks (DSBs); separately each domain can produce single-stranded breaks. Base-pairing between the gRNA and target nucleic acid causes double-stranded breaks (DSBs) due to the endonuclease activity of Cas9. Binding specificity is determined by both gRNA-target nucleic acid base pairing and the PAM juxtaposed to the DNA complementary region. In particular embodiments, the CRISPR system only requires a minimal set of two molecules—the Cas protein and the gRNA.

A large number of Cas9 orthologs are known in the art (Fonfara et al., NAR, 42:2577-2590, 2014; Chylinski et al. NAR, 42:6091-6105, 2014; Esvelt et al. Nature Methods, 10:1116-1121, 2013). A number of orthogonal Cas9 proteins have been identified including Cas9 proteins from Neisseria meningitidis, Streptococcus thermophilus and Staphylococcus aureus. Other Class 2 Cas proteins that can be used include Cas12a (Cpf1), Cas13a (C2c2), and Cas13B (C2c6). The Cpf1 nuclease particularly can provide added flexibility in target site selection by means of a short, three base pair recognition sequence (TTN), known as the protospacer-adjacent motif or PAM. Cpf1's cut site is at least 18 bp away from the PAM sequence, thus the enzyme can repeatedly cut a specified locus after indel (insertion and deletion) formation. Exemplary engineered Cpf1s are described in US 2018/0030425, US 2016/0208243, WO/2017/184768 and Zetsche et al., Cell 163: 759-771, 2015; and single gRNAs in Jinek et al., Science 337:816-821, 2012; Jinek et al., eLife 2:e00471, 2013; Segal, eLife 2:e00563, 2013.

In particular embodiments, polynucleotide sequences encoding mutant forms of Cas9 nuclease can be used in genetic constructs of the disclosure. For example, a Sniper Cas9, a variant of Cas9 with optimized specificity (minimal off-target effects) and retained on-target activity can be used (Lee et al., J Vis Exp. (144), 2019; Lee et al., Nat Commun. 9(1):3048, 2018; WO 2017/217768). As another example, a mutant Cas9 nuclease containing a D10A amino acid substitution can be used. This mutant Cas9 has lost double-stranded nuclease activity present in the wild type Cas9 but retains partial function as a single-stranded nickase. This mutant Cas9 generates a break in the complementary strand of DNA rather than both strands. This allows repair of the DNA template using a high-fidelity pathway rather than non-homologous end joining (NHEJ). The higher fidelity pathway prevents formation of insertions/deletions at the targeted locus while maintaining ability to undergo homologous recombination (Cong et al., Science 339(6121):819-823, 2013). Paired nicking has been shown to reduce off-target activity by 50- to 1,500-fold in cell lines (Ran et al., Cell 154(6):1380-1389, 2013).

In particular embodiments, a Cas protein can include one or more degrons to self-inactivate the Cas protein by accelerating degradation of expressed Cas protein. A degron can include a portion of a polypeptide that is important in regulation of protein degradation. In particular embodiments, a degron includes short amino acid sequences, structural motifs, and/or exposed amino acids (e.g., a lysine or arginine) located anywhere in a protein. In particular embodiments, a degron can be ubiquitin-dependent or ubiquitin-independent.

In particular embodiments, a Cas protein can be fused to a heterologous polypeptide that provides for subcellular localization. Such heterologous peptides include, for example, a nuclear localization signal (NLS) such as the SV40 NLS for targeting to the nucleus (e.g., see Lange et al., J. Biol. Chem. 282:5101-5105, 2007). Such subcellular localization signals can be located at the N-terminus, the C-terminus, or anywhere within the Cas protein. An NLS can include a stretch of basic amino acids and can be a monopartite sequence or a bipartite sequence.

In particular embodiments, a Cas protein can also include a heterologous polypeptide for ease of tracking or purification, such as a fluorescent protein, a purification tag, or an epitope tag. Examples of tags include green fluorescent protein (GFP), glutathione-S-transferase (GST), myc, Flag, hemagglutinin (HA), Nus, Softag 1, Softag 3, Strep, polyhistidine, biotin carboxyl carrier protein (BCCP), maltose binding protein (MBP), and calmodulin.

Additional information regarding CRISPR-Cas systems and components thereof are described in U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233, 8,999,641, and applications related thereto; and International Patent Publications WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473, WO2015/089486, WO2016/205711, WO2017/106657, WO2017/127807, and applications related thereto.

Teachings of the disclosure in relation to CRISPR can be applied to other gene editing systems that similarly utilize nucleases.

Embodiments utilize zinc finger nucleases (ZFNs) as gene editing agents. ZFNs are a class of site-specific nucleases engineered to bind and cleave DNA at specific positions. ZFNs are used to introduce double strand breaks (DSBs) at a specific site in a DNA sequence which enables the ZFNs to target unique sequences within a genome in a variety of different cells. Moreover, subsequent to double-stranded breakage, homology-directed repair (HDR) or non-homologous end joining (NHEJ) takes place to repair the DSB, thus enabling genome editing.

ZFNs are synthesized by fusing a zinc finger DNA-binding domain to a DNA cleavage domain. The DNA-binding domain includes three to six zinc finger proteins which are transcription factors. The DNA cleavage domain includes the catalytic domain of, for example, Fokl endonuclease. The Fokl domain functions as a dimer requiring two constructs with unique DNA binding domains for sites on the target sequence. The Fokl cleavage domain cleaves within a five or six base pair spacer sequence separating the two inverted half-sites.

Additional information regarding ZFNs can be found, for instance, in Kim et al., PNAS USA 93:1156-1160, 1996; Wolfe et al., Ann Rev Biophysics Biomol Struct. 29:183-212, 2000; Bibikova et al., Science 300:764, 2003; Bibikova et al., Genetics 161:1169-1175, 2002; Miller et al., EMBO J. 4:1609-1614, 1985; and Miller, et al. Nature Biotech 25:778-785, 2007.

Embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing DSBs in the DNA, which induce repair mechanisms in cells. Generally, two TALENs must bind and flank each side of the target DNA site for the DNA cleavage domain to dimerize and induce a DSB. The DSB is repaired in the cell by NHEJ or HDR if an exogenous double-stranded donor DNA fragment is present.

Additional gene editing agents include transcription activator-like effector nucleases (TALENs). TALENs refer to fusion proteins including a transcription activator-like effector (TALE) DNA binding protein and a DNA cleavage domain. TALENs are used to edit genes and genomes by inducing double strand breaks (DSBs) in the DNA, which induce repair mechanisms in cells. Generally, two TALENs must bind and flank each side of the target DNA site for the DNA cleavage domain to dimerize and induce a DSB. The DSB is repaired in the cell by non-homologous end-joining (NHEJ) or by homologous recombination (HR) with an exogenous double-stranded donor DNA fragment.

As indicated, TALENs have been engineered to bind a target sequence of, for example, an endogenous genome, and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12th and 13th positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

Examples of DNA cleavage domains that can be used in TALEN fusions are wild-type and variant Fokl endonucleases. The Fokl domain functions as a dimer requiring two constructs with unique DNA binding domains for sites on the target sequence. The Fokl cleavage domain cleaves within a five or six base pair spacer sequence separating the two inverted half-sites.

Particular embodiments utilize MegaTALs as gene editing agents. MegaTALs have a single chain rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Yet another embodiment that provides a Cannabis plant with a nullified THC pathway involves altering (disabling, removing, or redirecting) the signal peptide naturally found on THCA synthase. THCA is cytotoxic and causes apoptosis to its own plant cells, so THCA synthase is produced and secreted extracellularly by secretory cells into the storage cavity of the glandular trichome, where it becomes active and produces THCA+H₂O₂ from CBGA substrate, perhaps as a plant defense against pests (Sirikantaramas et al., Plant Cell Physiol 46(9):1578-1582, 2005). If a cell can be prevented from exporting THC synthase from the secretory cell, for instance redirecting it to a storage cavity (such as the vacuole), then the protein would not become active and THCA production would be reduced or eliminated. For instance, U.S. Patent Publication No. 2019/0338301 describes that THCA synthase contains a 28 amino acid signal peptide that directs its export out of the cell and into the extracellular trichome. This signal peptide can be disabled to prevent extracellular export (and therefore functional activation) of the THCA synthase. Alternatively, the signal peptide can be substituted with a different targeting sequence, such as one that would target the protein to the vacuole or to another subcellular location within the plant cell. See, for instance, Pereira et al. (Int J Mol Sci 15(5):7611-7623, 2014; describing delivery of proteins to the plant vacuole); Nakamura & Matsuoka (Plant Physiol. 101(1):1-5, 1993); Matsuoka & Neuhaus (J Exper Botany, 50(331):165-174, 1999; discussing cis-elements involved in protein transport to plant vacuoles); and Park et al. (Plant Physiol. 134(2):625-639, 2004).

### IV. CBDAS-Inhibitory Nucleic Acid Molecules

Also described herein are nucleic acid molecules useful for decreasing the expression of the CBDAS gene in Cannabis. Methodologies similar to those described above to reduce the expression THCAS and CBCAS in Cannabis are entirely amenable to target expression of CBDAS, in order to produce plants that have a higher level of THC and/or CBC.

As described herein, the CBDAS sequence motif shown in SEQ ID NO: 103 is highly conserved across myriad Cannabis strains; it is considered unique, as it is different from the THCAS/CBCAS motif described herein. The sequence in SEQ ID NO: 103 can be selectively targeted with an antisense polyribonucleotide in a hpRNA (or targeted with CRISPR) which targets the highly-conserved motif, in order to selectively downregulate expression of the CBDAS gene in a Cannabis plant. Optionally, this sequence may be used to generate a null CBDAS phenotype, for instance using CRISPR modification of the CBDAS gene, targeted specifically to the sequence in SEQ ID NO: 103.

### V. Genetic Modifications in Cannabis

Recombinant nucleic acid molecules, as described, supra, may be used in methods for the creation of genetically modified Cannabis plant materials, and for the expression of hpRNAs of the disclosure to produce transgenic Cannabis plants with modified chemotypes; for example, THC- and CBC-null Cannabis plants, or THC and/or CBC enhanced Cannabis plants. Such transgenic plants may be prepared, for example, by inserting polynucleotides encoding the hpRNA molecules into plant transformation vectors, and introducing these into Cannabis plants or plant materials, from which viable plants may in particular embodiments by regenerated.

In some embodiments, a method for producing a genetically modified Cannabis plant material is provided, wherein the method comprises introducing into a Cannabis plant material at least one nucleic acid molecule comprising a polynucleotide of the disclosure. In particular embodiments, the method comprises transforming a Cannabis plant cell (for example, in a cell culture, tissue culture, or callus culture) with the nucleic acid molecule to produce a transformed Cannabis plant cell, and culturing the transformed plant cell under conditions sufficient to allow for development of a plant cell culture comprising a plurality of genetically modified plant cells, selecting for genetically modified plant cells that have integrated the polynucleotide into their genomes, screening the genetically modified plant cells for expression of the hpRNA molecule encoded by the polynucleotide, and selecting a genetically modified plant cell that expresses the dsRNA. The selected genetically modified Cannabis plant cell may be cultured under conditions sufficient to allow for the development of a genetically modified Cannabis plant material consisting essentially of genetically modified Cannabis plant cells. The genetically modified Cannabis plant material may be regenerated to produce a transgenic Cannabis plant comprising the polynucleotide.

In other embodiments, the method comprises crossing a transgenic Cannabis plant comprising a polynucleotide of the disclosure with a different Cannabis plant to produce a population of progeny plants, screening the population of progeny plants for presence of the polynucleotide or expression of the hpRNA encoded by the polynucleotide, and selecting a progeny transgenic Cannabis plant comprising the polynucleotide. The selected progeny transgenic Cannabis plant may be subsequently selfed or backcrossed with the different Cannabis plant for one or more generations, for example, to produce an inbred transgenic Cannabis plant comprising the polynucleotide. In these and further embodiments, the selected progeny transgenic Cannabis plant may be itself cultivated or utilized to produce a hybrid plant.

Suitable methods herein for transformation of Cannabis plant materials include any method by which DNA can be introduced into a plant cell, such as by transformation of protoplasts (see, e.g., U.S. Pat. No. 5,508,184), by desiccation/inhibition-mediated DNA uptake (see, e.g., Potrykus et al., Mol. Gen. Genet. 199:183-8, 1985), by electroporation (see, e.g., U.S. Pat. No. 5,384,253), by agitation with silicon carbide fibers (see, e.g., U.S. Pat. Nos. 5,302,523 and 5,464,765), by Agrobacterium-mediated transformation (see, e.g., U.S. Pat. Nos. 5,563,055; 5,591,616; 5,693,512; 5,824,877; 5,981,840; and 6,384,301) and by acceleration of DNA-coated particles (see, e.g., U.S. Pat. Nos. 5,015,580; 5,550,318; 5,538,880; 6,160,208; 6,399,861; and 6,403,865). Through the application of techniques such as these, the cells of virtually any plant, including Cannabis species, may be stably transformed. In some embodiments, transformation results in integration of a heterologous polynucleotide into the genome of the host cell. Any of these techniques may be used to produce a transgenic Cannabis plant material, for example, comprising one or more polynucleotides encoding hpRNA molecules in the genome of the transgenic plant material.

Exemplary techniques for transforming Cannabis plants are described in Deguchi et al., Scientific Reports 10:3504, 2020 (doi.org/10.1038/s41598-020-60323-9); and in Zhang et al., Plant Biotech J., 1-20, 2021 (doi.org/10.1111/pbi.13611).

The most widely utilized method for introducing an expression vector into plants is based on the natural transformation system of Agrobacterium. A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. The Ti (tumor-inducing)-plasmids contain a large segment, known as T-DNA, which is transferred to transformed plants. Another segment of the Ti plasmid, the Vir region, is responsible for T-DNA transfer. The T-DNA region is bordered by terminal repeats. In modified binary vectors, the tumor-inducing genes have been deleted, and the functions of the Vir region are utilized to transfer foreign DNA bordered by the T-DNA border elements. The T-region may also contain a selectable marker for efficient recovery of genetically modified cells and plants, and a multiple cloning site for inserting polynucleotides for transfer such as a dsRNA encoding nucleic acid.

Thus, in some embodiments, a plant transformation vector is derived from a Ti plasmid of A. tumefaciens (See, e.g., U.S. Pat. Nos. 4,536,475, 4,693,977, 4,886,937, and 5,501,967; and European Patent No. EP 0 122 791) or a Ri plasmid of A. rhizogenes. Additional plant transformation vectors include, for example and without limitation, those described by Herrera-Estrella et al., Nature 303:209-13, 1983; Bevan et al., Nature 304:184-7, 1983; Klee et al., Bio/Technol. 3:637-42, 1985; and in European Patent No. EP 0 120 516, and those derived from any of the foregoing. Other bacteria such as Sinorhizobium, Rhizobium, and Mesorhizobium that interact with plants naturally can be modified to mediate gene transfer to a number of diverse plants. These plant-associated symbiotic bacteria can be made competent for gene transfer by acquisition of both a disarmed Ti plasmid and a suitable binary vector.

After transforming recipient cells with a heterologous polynucleotide encoding an hpRNA molecule of the disclosure, transformed cells are generally identified for further culturing and plant regeneration. In order to improve the ability to identify transformed cells, one may desire to employ a selectable or screenable marker gene, as previously set forth, with the transformation vector used to generate the transformant. In the case where a selectable marker is used, transformed cells are identified within the potentially transformed cell population by exposing the cells to a selective agent or agents. In the case where a screenable marker is used, cells may be screened for the desired marker gene trait.

Cells that survive the exposure to the selective agent, or cells that have been scored positive in a screening assay, may be cultured in media that supports regeneration of plants. In some embodiments, any suitable plant tissue culture media (e.g., MS and N6 media) may be modified by including further substances, such as growth regulators. Tissue may be maintained on a basic medium with growth regulators until sufficient tissue is available to begin plant regeneration efforts, or following repeated rounds of manual selection, until the morphology of the tissue is suitable for regeneration (e.g., at least 2 weeks), then transferred to media conducive to shoot formation. Cultures are transferred periodically until sufficient shoot formation has occurred. Once shoots are formed, they are transferred to media conducive to root formation. Once sufficient roots are formed, plants can be transferred to soil for further growth and maturation.

To confirm the presence of the polynucleotide encoding the THCAS/CBCAS-inhibitory hpRNA molecule(s) in regenerating plants, a variety of assays may be performed. Such assays include, for example: molecular biological assays, such as Southern and northern blotting, PCR, and nucleic acid sequencing; biochemical assays, such as detecting the presence of a THCAS and/or CBCAS, e.g., by immunological means (ELISA and/or western blots) or by enzymatic function; plant part assays, such as leaf or root assays; and analysis of the phenotype of the whole regenerated plant.

Integration events may be analyzed, for example, by polymerase-chain reaction (PCR) amplification using, e.g., oligonucleotide primers specific for a polynucleotide of interest. PCR genotyping is understood to include, but not be limited to, PCR amplification of gDNA derived from isolated host plant callus tissue predicted to contain a polynucleotide of interest integrated into the genome, followed by standard cloning and sequence analysis of PCR amplification products. Methods of PCR genotyping have been well described (for example, Rios et al., Plant J. 32:243-53, 2002) and may be applied to gDNA derived from and Cannabis plant or tissue type, including cell cultures.

A transgenic plant formed using Agrobacterium-dependent transformation methods typically contains a single recombinant DNA inserted into one chromosome. The polynucleotide of the single recombinant DNA is referred to as a “transgenic event” or “integration event.” Such transgenic plants are heterozygous for the inserted heterologous polynucleotide. In some embodiments, a transgenic plant homozygous with respect to the hpRNA-encoding polynucleotide may be obtained by sexually mating (selfing) an independent segregant transgenic plant that contains a single hpRNA-encoding polynucleotide to itself, for example a T₀ plant, to produce T₁ seed. One fourth of the T₁ seed produced will be homozygous with respect to the hpRNA-encoding polynucleotide. Germinating Ti seed results in plants that can be tested for heterozygosity, typically using an SNP assay or a thermal amplification assay that allows for the distinction between heterozygotes and homozygotes (i.e., a zygosity assay).

In addition to direct transformation of a Cannabis plant with a recombinant nucleic acid molecule, a hpRNA-encoding polynucleotide may be introduced into a Cannabis plant by plant-to-plant transmission. In some embodiments, a transgenic Cannabis plant is produced by crossing a first plant having at least one transgenic event with a second plant lacking such an event. In specific examples, a hpRNA-encoding polynucleotide may be introduced into a first plant line that is amenable to transformation to produce a transgenic plant comprising the polynucleotide, which transgenic plant may subsequently be crossed with a second plant line to produce a progeny plant comprising the polynucleotide. By backcrossing the plant produced by the cross with the second plant line and selecting for progeny plants comprising the polynucleotide through multiple generations, the polynucleotide is introgressed into the second plant line, yielding a transgenic Cannabis plant comprising the polynucleotide and a minimal amount of surrounding gDNA from the first plant line otherwise in the genetic background of the second plant line, for example, such that the introgressed plant comprises the hpRNA-encoding polynucleotide and a chemotype corresponding to the presence in the plant of the hpRNA, and any desirable characteristics of the second plant line. In other specific examples, a Cannabis plant comprising a polynucleotide encoding a hpRNA molecule of the disclosure is used to pollinate a second Cannabis plant that does not comprise the polynucleotide, or vice versa. Once a polynucleotide encoding a hpRNA molecule of the disclosure is introduced into a Cannabis plant by any method, the Cannabis plant may be backcrossed to produce an inbred variety.

### VI. Genetically Modified Cannabis Plants and Plant Parts/Plant Materials

Some embodiments herein include a genetically modified Cannabis plant material; i.e., a Cannabis plant material comprising a polynucleotide encoding a hpRNA molecule (for example, integrated in the genome), wherein the plant material comprises the hpRNA molecule encoded by the polynucleotide. In particular embodiments, the genetically modified Cannabis plant material comprises a polynucleotide comprising a nucleotide sequence that encodes an antisense polyribonucleotide of an hpRNA molecule, comprising a nucleotide sequence that is substantially identical to the complement or reverse complement of any of SEQ ID NOs: 33-89, wherein the nucleotide sequence comprises at least 12 contiguous nucleotides of the complement or reverse complement of the motif defined by SEQ ID NO: 1 and SEQ ID NOs: 2-29 (for example, the motif of any of SEQ ID NOs: 17, 19, 24, 28, 43, and 80). In these and other particular embodiments, the polynucleotide may comprise a nucleotide sequence encoding an antisense polyribonucleotide of the hpRNA molecule, wherein the nucleotide sequence is at least 90% identical to the complement or reverse complement of the motif defined by SEQ ID NO: 1 and SEQ ID NOs: 2-29; for example, the motif of any of SEQ ID NOs: 17, 19, 24, 28, 43, and 80.

In particular embodiments, the polynucleotide may comprise a nucleotide sequence encoding an antisense polyribonucleotide of the hpRNA molecule, wherein the nucleotide sequence is substantially identical to any of SEQ ID NOs: 33-89, wherein the nucleotide sequence comprises at least 12 contiguous nucleotides of the motif defined by SEQ ID NO: 1 and SEQ ID NOs: 2-29 (for example, the motif of any of SEQ ID NOs: 17, 19, 24, 28, 43, and 80). In these and other particular embodiments, the polynucleotide may comprise a nucleotide sequence encoding an antisense polyribonucleotide of the hpRNA molecule, wherein the nucleotide sequence is at least 90% identical to the motif defined by SEQ ID NO: 1 and SEQ ID NOs: 2-29; for example, the motif of any of SEQ ID NOs: 17, 19, 24, 28, 43, and 80. In some embodiments, the polynucleotide encoding the hpRNA molecule is selected from the group consisting of SEQ ID NOs: 128-138; for example, any of SEQ ID NOs: 131-138 (e.g., SEQ ID NO: 137).

A genetically modified Cannabis plant material comprising a polynucleotide encoding a hpRNA molecule may also comprise the hpRNA molecule encoded by the polynucleotide in one or more tissues wherein expression of the hpRNA is initiated by an operably linked promoter. In particular embodiments, expression of the hpRNA molecule in the plant material is sufficient to decrease or substantially eliminate the expression of the target THCAS/CBCAS genes in the plant material, such that production of THCA and/or CBCA is reduced or substantially eliminated. Consequently, the genetically modified Cannabis plant materials described herein may display modified chemotypes that are expected from the decreased amounts of THCA/THC and/or CBCA/CBC (for example, a THC-null and CBC-null chemotype), and the related increase in production of other cannabinoids, such as CBD, and small molecules resulting from diversion of CBGA substrate into alternative cellular biosynthetic pathways.

In embodiments, expression of the target THCAS/CBCAS genes in the plant material may be inhibited by at least 10%, at least 20%, at least 30%, at least 40%, at least 50%, at least 60%, at least 70%, at least 80%, at least 90%, at least 95%, at least 96%, at least 97%, at least 98%, at least 99%, or about 100% within the plant material, such that a significant inhibition of production of THCA and/or CBCA occurs. Significant inhibition refers to inhibition over a threshold that results in a detectable change in the chemotype of the Cannabis plant material. This specifically means that the expression of the target THCAS/CBCAS genes in the plant material may be inhibited by more than any numerical amount between 50% and 100%; for example, more than 75%, more than 80%, more than 85%, more than 90%, more than 90.5%, more than 91%, more than 91.5%, more than 92%, more than 92.5%, more than 93%, more than 93.5%, more than 94%, more than 94.5%, more than 95%, more than 95.5%, more than 96%, more than 96.5%, more than 97%, more than 97.5%, more than 98%, more than 98.5%, more than 99%, or more than 99.5%. While in certain examples, inhibition occurs in substantially all cells and tissues of the plant material expressing the target THCAS/CBCAS genes, in other embodiments inhibition occurs only in a subset of cells and tissues expressing the target THCAS/CBCAS genes.

The above description applies also to production of Cannabis plants in which CBDAS expression is inhibited (while expression of THCAS and CBCAS genes is not significantly impacted).

In some embodiments, a transgenic Cannabis plant material having in its genome at least one recombinant polynucleotide encoding an hpRNA molecule, or a genomic sequence that has been intentionally modified using CRISPR, of the disclosure may be a transformed plant material (for example, a cell, tissue, cell culture, or tissue culture). In addition to such transformed plant materials, progeny plants of any transgenic plant generation, transgenic seeds, and transgenic plant products, are all provided, each of which comprises the recombinant polynucleotide encoding the hpRNA molecule (or CRISPR modified nucleotide). In particular embodiments, the hpRNA molecule may be expressed in the foregoing transgenic plants, seeds, and plant products. Therefore, in these and other embodiments, the hpRNA molecule or a polynucleotide sequence that encodes it may be isolated from a genetically modified or transgenic Cannabis plant, plant material, or a plant product (such as a plant commodity product) of the disclosure. In particular examples, the genetically modified Cannabis plant, plant material, or plant product is of a Cannabis species selected from the group consisting of hemp-type or fiber-type Cannabis species. In other examples, the genetically modified Cannabis plant material or plant product is of a Cannabis species selected from the group consisting of intoxicant Cannabis species; e.g., high-THC Cannabis species, high-CBD Cannabis species, high-CBC Cannabis species, and Cannabis species that have been genetically modified or produced by conventional breeding methods to comprise specific ratios of THC:CBD, THC:CBC, CBC:CBD, or THC:CBD:CBC.

Particular embodiments include methods for producing a transgenic Cannabis plant comprising a modified chemotype, wherein the methods include cultivating a transgenic Cannabis plant comprising a polynucleotide encoding an hpRNA molecule of the disclosure.

Seeds produced by transgenic Cannabis plant materials comprising a recombinant polynucleotide encoding an hpRNA molecule of the disclosure, or encoding a genomic sequence that has been intentionally modified using CRISPR, and plant commodity products derived from genetically modified Cannabis plant materials, including such transgenic Cannabis plant materials or seeds thereof, are therefore specifically described herein, wherein the seeds or commodity products comprise a detectable amount of a polynucleotide or encoded hpRNA. It is recognized that some commodity products derived or produced from the transgenic plants/plant materials may not have significant amounts of polynucleotide/hpRNA (such as oils/extracts, where just the composition predominantly includes cannabinoids but other material from the Cannabis plant/plant material has been removed).

Use of such a Cannabis plant material or seed in the production of a plant commodity product may be desirable, for example, to utilize the modified chemotype exhibited by the Cannabis plant material or seed, whether that chemotype is reflected in the final commodity product or not (where, for example, the composition of the commodity product is subsequently altered to comprise a different cannabinoid content than the Cannabis plant materials Cannabis plant material or seed). In some embodiments, such commodity products may be produced, for example, by cultivating or otherwise obtaining a transgenic Cannabis plant comprising the polynucleotide and/or hpRNA, and preparing the commodity product by isolating a part of or the entire transgenic Cannabis plant, and subsequently processing that plant/plant part according to methods known in the art. Specific commodity products comprising (perhaps at only trace levels) one or more of the polynucleotides and/or hpRNA molecules of the disclosure include, for example and without limitation: food products (for example, baked goods (e.g., cookies), beverages (e.g., coffee and soda), candy, and consumable oils, extracts, and concentrates), inhalable products (e.g., cigarettes and vape oils), concentrates (e.g., for use in vaporizers), creams (e.g., face creams and tattoo creams), extracts, flower, hemp, fiber, oils (e.g., body oils, beard oils, and massage oils), medicaments (e.g., Epidiolex™ and Sativex™), salves, ointments, cosmetics, soaps, lip balms, hair products (e.g., shampoos), bath bombs, bath salts, gels (e.g., topical gels), lotions, roll-on skin products and deodorants, patches (e.g., topical patches and transdermal patches), capsules, tablets, strips (e.g., oral, dissolving strips), and any of the foregoing, formulated for human use or use by animals (e.g., pets). Both over the counter and pharmaceutical preparations are specifically contemplated.

The detection of a polynucleotide or hpRNA of the current disclosure in one or more commodity products is de facto evidence that the commodity product is produced from a genetically modified Cannabis plant material of the disclosure.

All references, including publications, patents, patent applications, and sequence entries in public databases, cited herein are hereby incorporated by reference to the extent they are not inconsistent with the explicit details of this disclosure, and are so incorporated to the same extent as if each reference were individually and specifically indicated to be incorporated by reference and were set forth in its entirety herein. If the reference occurs in different versions, or is subject to being updated over time, the version that is incorporated by reference is the one that was most recently publicly available on the filing date of the first application in which the reference is cited.

The Exemplary Embodiments and Examples below are included to demonstrate particular embodiments of the disclosure, and to illustrate certain particular features. They should not be construed to limit the disclosure to the particular features or embodiments exemplified. Those of ordinary skill in the art will recognize, in light of the present disclosure, that many changes can be made to the specific embodiments disclosed herein and still obtain a like or similar result without departing from the spirit and scope of the disclosure.

### VII. Exemplary Embodiments

1. A nucleic acid molecule including at least one polynucleotide operably linked to a plant promoter that functions in a Cannabis plant, wherein the polynucleotide encodes a hairpin RNA (hpRNA) molecule, and wherein the polynucleotide includes: a first nucleotide sequence encoding a first polyribonucleotide in the hpRNA molecule, wherein the first nucleotide sequence is between 20 and 30 nucleotides in length and is substantially identical to the complement or reverse complement of a Cannabis THCAS/CBCAS gene, and the first nucleotide sequence includes at least 12 contiguous nucleotides of the complement or reverse complement of SEQ ID NO: 17 or SEQ ID NO: 28, and a second nucleotide sequence encoding a sense polyribonucleotide in the hpRNA molecule that is substantially the reverse complement of the first nucleotide sequence, wherein the first and second nucleotide sequence are separated in the polynucleotide by a nucleotide sequence that encodes a loop structure in the hpRNA molecule.
2. A nucleic acid molecule including at least one polynucleotide operably linked to a plant promoter that functions in a Cannabis plant, wherein the polynucleotide encodes a hairpin RNA (hpRNA) molecule, and wherein the polynucleotide includes: a first nucleotide sequence encoding a first polyribonucleotide in the hpRNA molecule, wherein the first nucleotide sequence is between 20 and 30 nucleotides in length and is substantially identical to the complement or reverse complement of a Cannabis THCAS/CBCAS gene, and is selected from the group consisting of: a nucleotide sequence that includes at least 12 contiguous nucleotides of the complement or reverse complement of the motif defined by SEQ ID NO: 1 and SEQ ID NOs: 2-29, preferably wherein the nucleotide sequence includes at least 12 contiguous nucleotides of the complement or reverse complement of a nucleotide sequence selected from the group consisting of SEQ ID NOs: 17, 19, 24, 28, 43, and 80, most preferably wherein the nucleotide sequence includes at least 12 contiguous nucleotides of the complement or reverse complement of SEQ ID NO: 17 or SEQ ID NO: 28, and a nucleotide sequence that is at least 80% identical over its length to the complement or reverse complement of the motif defined by SEQ ID NO: 1 and SEQ ID NOs: 2-29, preferably wherein the nucleotide sequence is at least 80% identical over its length to the complement or reverse complement of a nucleotide sequence selected from the group consisting of SEQ ID NOs: 17, 19, 24, 28, 43, and 80, most preferably wherein the nucleotide sequence is at least 80% identical over its length to the complement or reverse complement of SEQ ID NO: 17 or SEQ ID NO: 28, even more preferably wherein the nucleotide sequence is at least 90% identical over its length to the complement or reverse complement of the motif defined by SEQ ID NO: 1 and SEQ ID NOs: 2-29, preferably wherein the nucleotide sequence is at least 90% identical over its length to the complement or reverse complement of a nucleotide sequence selected from the group consisting of SEQ ID NOs: 17, 19, 24, 28, 43, and 80, most preferably wherein the nucleotide sequence is at least 90% identical over its length to the complement or reverse complement of SEQ ID NO: 17 or SEQ ID NO: 28; a second nucleotide sequence encoding a sense polyribonucleotide in the hpRNA molecule that is substantially the reverse complement of the first nucleotide sequence, wherein the first and second nucleotide sequence are separated in the polynucleotide by a nucleotide sequence that encodes a loop structure in the hpRNA molecule.
3. A nucleic acid molecule including at least one polynucleotide operably linked to a plant promoter that functions in a Cannabis plant, wherein the polynucleotide encodes a hairpin RNA (hpRNA) molecule, and wherein the polynucleotide includes: a first nucleotide sequence encoding a first polyribonucleotide in the hpRNA molecule, wherein the first nucleotide sequence is between 20 and 30 nucleotides in length and is substantially identical to the complement or reverse complement of a Cannabis THCAS/CBCAS gene, and is selected from the group consisting of: a nucleotide sequence that includes at least 12 contiguous nucleotides of the complement or reverse complement of the motif defined by SEQ ID NO: 1 and SEQ ID NOs: 2-29, and a nucleotide sequence that is at least 80% identical over its length to the complement or reverse complement of the motif defined by SEQ ID NO: 1 and SEQ ID NOs: 2-29, a nucleotide sequence is at least 90% identical over its length to the complement or reverse complement of the motif defined by SEQ ID NO: 1 and SEQ ID NOs: 2-29; a second nucleotide sequence encoding a sense polyribonucleotide in the hpRNA molecule that is substantially the reverse complement of the first nucleotide sequence, wherein the first and second nucleotide sequence are separated in the polynucleotide by a nucleotide sequence that encodes a loop structure in the hpRNA molecule.
4. The nucleic acid molecule of embodiment 3, wherein the first nucleotide sequence includes: at least 12 contiguous nucleotides of the complement or reverse complement of a nucleotide sequence selected from the group consisting of SEQ ID NOs: 17, 19, 24, 28, 43, and 80; or at least 12 contiguous nucleotides of the complement or reverse complement of SEQ ID NO: 17 or SEQ ID NO: 28.
5. The nucleic acid molecule of embodiment 3, wherein the first nucleotide sequence is: at least 80% identical over its length to the complement or reverse complement of a nucleotide sequence selected from the group consisting of SEQ ID NOs: 17, 19, 24, 28, 43, and 80; or at least 80% identical over its length to the complement or reverse complement of SEQ ID NO: 17 or SEQ ID NO: 28; or at least 90% identical over its length to the complement or reverse complement of the motif defined by SEQ ID NO: 1 and SEQ ID NOs: 2-29; or at least 90% identical over its length to the complement or reverse complement of a nucleotide sequence selected from the group consisting of SEQ ID NOs: 17, 19, 24, 28, 43, and 80; or at least 90% identical over its length to the complement or reverse complement of SEQ ID NO: 17 or SEQ ID NO: 28.
6. The nucleic acid molecule of any one of embodiments 1-5, wherein the molecule is a plant transformation vector or contained within a plant transformation vector.
7. A genetically modified Cannabis plant material or commodity product made of or from a Cannabis plant including the polynucleotide from the nucleic acid molecule of any one of embodiments 1-5 or the hpRNA molecule encoded by the polynucleotide.
8. The genetically modified Cannabis plant material or commodity product made of or from a Cannabis plant of embodiment 7, wherein the Cannabis plant is C. sativa, C. indica (non-hybrid), a C. sativa/C. indica hybrid, or C. sativa subspecies, C. ruderalis.
9. The genetically modified Cannabis plant material or commodity product made of or from a Cannabis plant of embodiment 7 or embodiment 8, wherein the plant material is a transgenic Cannabis plant including the polynucleotide stably integrated in its genome operably linked to the promoter.
10. A vector configured to express the nucleic acid molecule of any one of embodiments 1-5.
11. A cell derived from the genetically modified Cannabis plant of embodiment 7 or embodiment 8, which cell includes the hpRNA molecule or a nucleic acid molecule encoding it.
12. A method of making a genetically modified Cannabis plant, including: transforming a cell of a Cannabis plant with the vector of embodiment 10 to produce a transformed cell; regenerating a plant from the transformed cell, which plant is the genetically modified Cannabis plant.
13. A nucleic acid molecule including at least one polynucleotide operably linked to a plant promoter that functions in a Cannabis plant, wherein the polynucleotide encodes a hairpin RNA (hpRNA) molecule, and wherein the polynucleotide includes: a first nucleotide sequence encoding a first polyribonucleotide in the hpRNA molecule, wherein the first nucleotide sequence is between 20 and 30 nucleotides in length and is substantially identical to the complement or reverse complement of a Cannabis CBDAS gene, and the first nucleotide sequence includes at least 12 contiguous nucleotides of the complement or reverse complement of SEQ ID NO: 103, and a second nucleotide sequence encoding a sense polyribonucleotide in the hpRNA molecule that is substantially the reverse complement of the first nucleotide sequence, wherein the first and second nucleotide sequence are separated in the polynucleotide by a nucleotide sequence that encodes a loop structure in the hpRNA molecule.
14. A nucleic acid molecule including at least one polynucleotide operably linked to a plant promoter that functions in a Cannabis plant, wherein the polynucleotide encodes a hairpin RNA (hpRNA) molecule, and wherein the polynucleotide includes: a first nucleotide sequence encoding a first polyribonucleotide in the hpRNA molecule, wherein the first nucleotide sequence is between 20 and 30 nucleotides in length and is substantially identical to the complement or reverse complement of a Cannabis CBDAS gene, and is selected from the group consisting of: a nucleotide sequence that includes at least 12 contiguous nucleotides of the complement or reverse complement of the motif defined by SEQ ID NO: 103, a nucleotide sequence that is at least 80% identical over its length to the complement or reverse complement of the motif defined by SEQ ID NO: 103, and a nucleotide sequence is at least 90% identical over its length to the complement or reverse complement of the motif defined by SEQ ID NO: 103; a second nucleotide sequence encoding a sense polyribonucleotide in the hpRNA molecule that is substantially the reverse complement of the first nucleotide sequence, wherein the first and second nucleotide sequence are separated in the polynucleotide by a nucleotide sequence that encodes a loop structure in the hpRNA molecule.
15. The nucleic acid molecule of embodiment 13 or embodiment 14, wherein the molecule is a plant transformation vector or contained within a plant transformation vector.
16. A genetically modified Cannabis plant material or commodity product made of or from a Cannabis plant including the polynucleotide from the nucleic acid molecule of any one of embodiments 13-15 or the hpRNA molecule encoded by the polynucleotide.
17. The genetically modified Cannabis plant material or commodity product made of or from a Cannabis plant of embodiment 16, wherein the Cannabis plant is C. sativa, C. indica (non-hybrid), a C. sativa/C. indica hybrid, or C. sativa subspecies, C. ruderalis.
18. The genetically modified Cannabis plant material or commodity product made of or from a Cannabis plant of embodiment 16 or embodiment 17, wherein the plant material is a transgenic Cannabis plant including the polynucleotide stably integrated in its genome operably linked to the promoter.
19. A vector configured to express the nucleic acid molecule of any one of embodiments 13-15.
20. A cell derived from the genetically modified Cannabis plant of embodiment 17 or embodiment 18, which cell includes the hpRNA molecule or a nucleic acid molecule encoding it.
21. A method of making a genetically modified Cannabis plant, including: transforming a cell of a Cannabis plant with the vector of embodiment 20 to produce a transformed cell; regenerating a plant from the transformed cell, which plant is the genetically modified Cannabis plant.
22. A method of modifying a Cannabis plant or plant cell using a CRISPR/Cas9 system, substantially as described herein.
23. The method of embodiment 22, wherein the CRISPR/Cas9 system is used to modify Cannabis THCAS/CBCAS gene(s) or to modify Cannabis CBDAS gene(s).
24. The method of embodiment 22 or embodiment 23, wherein modifying the Cannabis plant or plant cell relies on a consensus sequence as described herein.

### EXAMPLES

### Example 1: Genomics & Target Identification

Database sequence analysis and whole genome sequencing was performed on Cannabis species and cultivars to identify all genetic loci highly homologous to described THCAS, CBCAS, and CBDAS genes. Results of sequencing these cannabinoid synthases and comparing reported sequences annotated as THCAS, CBCAS, and CBDAS showed these genes are distributed in three distinct chromosomal regions. Twelve contigs (including SEQ ID NOs: 96-100) were found that contained copies of THCAS/CBCAS genes (ORFs of SEQ ID NOs: 90-95) in C. sativa subspecies, C. ruderalis (Finola). It was also found that there is a single copy of CBDAS (ORF of SEQ ID NO: 123).

Analysis of the CBCAS THCAS and CBDAS sequences from C. ruderalis, C. sativa, and C. sativa/indica hybrids revealed that all CBCAS, THCAS, and CBDAS include a homologous nucleotide sequence, as illustrated in FIGS. 2-3. Surprisingly, as shown in FIG. 3, these THCAS CBCAS and CBDAS sequences were found to be characterized and distinguished by a short (approximately 23-29 bp), highly homologous motif that is specifically conserved between THCAS and CBCAS (SEQ ID NOs: 30-89) (FIG. 3A), containing multiple characteristic polymorphisms with respect to the even more conserved CBDAS sequences (SEQ ID NOs: 109-122) (FIG. 3B).

Comparison of the C. ruderalis, C. sativa, and C. sativa/indica THCAS CBCAS and CBDAS sequences revealed that this homologous nucleotide sequence and its motif are indeed broadly conserved across the Cannabis genus. FIG. 3. Exhaustive analysis revealed that the highly homologous motif is unique in described CBCAS, THCAS, and CBDAS genes and the sequencing results shown in FIG. 3, distinguishing CBDAS from THCAS/CBCAS. It became apparent during the analysis that targeting other regions of THCAS/CBCAS would result in plants that would be highly susceptible to reactivation of the silenced gene by homologous recombination with other cannabinoid synthase genes in the genome (see Toth et al. (2020) GCB Bioenergy 12(3):213-22), such that stability of the null phenotype could not be maintained. The highly homologous motif was therefore selected as a particularly useful target for RNAi silencing of THCAS/CBCAS without silencing CBDAS to generate THCAS-null/CBCAS-null Cannabis.

The target motif sequence was found to contain characteristic polymorphisms (highlighted in black in FIGS. 2-3) that are conserved between NCBI database sequences and THCAS/CBCAS and CBDAS sequencing results from C. sativa, C. indica (non-hybrid), C. sativa/indica, and C. ruderalis, (FIG. 3), demonstrating the broad utility of our approach across members of the Cannabis genus.

FIG. 3A shows redundant sequencing data for THCAS/CBCAS as SEQ ID NOs: 33-83. Specific target motif sequences of interest corresponding to the most overlapping and repeating sequence data are defined by SEQ ID NOs: 33-44, SEQ ID NO: 77, and SEQ ID NO: 80. FIG. 3B shows redundant sequencing data for CBDAS as SEQ ID NOs: 33-83, containing the conserved targeting sequence of SEQ ID NO: 103. The presence of extremely conserved polymorphisms within the target motif sequences between the THCAS/CBCAS genes and the CBDAS gene allowed identification of siRNAs that are effective to selectively and stably silence THCAS/CBCAS expression, independent of CBDAS, by rational design of siRNA sequences that inactivate THCAS/CBCAS target sequences without being capable of inhibiting expression of CBDAS mRNA including the target sequence of SEQ ID NO: 103.

### Example 2: Target Modification

The highly homologous nucleotide sequence motif identified in Example 1 was used to engineer hpRNA-encoding constructs for RNAi silencing of THCAS CBCAS in Cannabis, resulting in a stable and significant reduction of THCAS and CBCAS activity. The constructs were designed with a polynucleotide encoding an hpRNA with a stem targeting THCAS/CBCAS sequence, SEQ ID NO: 28. As proof of concept, the SEQ ID NO: 28 targeting sequence was incorporated in a single polynucleotide encoding an hpRNA that also includes a stem sequence targeting a different THCAS/CBCAS sequence (SEQ ID NO: 17), where the two stem sequences are separated in the polynucleotide by a short linker on each hpRNA strand-forming sequence, and the by another hpRNA loop-forming linker between the two strand sequences. The resulting polynucleotide is represented by SEQ ID NO: 137. The polynucleotide was operably linked downstream to a C. sativa U6 promoter (SEQ ID NO: 139) to form the construct of SEQ ID NO: 140. This construct is particular useful for inactivating all THCAS/CBCAS mRNAs in C. sativa subspecies C. ruderalis (Finola), because together, the SEQ ID NO: 28 and SEQ ID NO: 17 siRNAs are substantially reverse complementary to all THCAS/CBCAS copies in this Cannabis variety.

### Example 3: Transformation of Cannabis

Cannabis varieties are transformed with expression constructs (such as that described in Example 2) containing SEQ ID NO: 28, including SEQ ID NO: 140, and whole plants are regenerated from the transformants. Cannabis plant materials comprising each of the constructs are phenotyped for cannabinoid composition, and predicted to have modified chemotypes as shown in Table 1. Untransformed plant values in Table 1 for THCA, CBCA, and CBDA were obtained from publicly-available sources; amounts are given with respect to dry plant weight.

TABLE 1
Predicted Changes in Cannabinoid Content in GM Cannabis Varieties.

| | | | | Predicted After |
|---|---|---|---|---|
| | Untransformed | | | Transformation* |
| Variety | THCA | CBC | CBDA | CBDA** |
| Cat | 0.3-0.72% | <0.05-0.1% | 0.14-14.25% | 0.44-15% |
| Lady | 3.08-7.19 mg/g | | 1.41-142.5 mg/g | 4.49-149.6 mg/g |
| The | 0.15-0.8% | ND | 4.06-19.73% | 4.22-20.63 |
| Wife | 1.49-8 mg/g | | 40.63-197.3 mg/g | 42-205 mg/g |
| Sweet | 0.31% | ND | 10.48% | 11% |
| Wife | 3.1 mg/g | | | 110 mg/g |
| Sweet | 0.63% | ND | 14.41% | 15% |
| Grass | 6.3 mg/g | | 144.1 mg/g | 150 mg/g |
| Superwoman | 0.75% | ND | 17.81% | 18.5% |
| | 7.5 mg/g | | 178.1 mg/g | 186 mg/g |
| Cherry | 0.27% | ND | 12.31% | 12.6% |
| wine | 2.7 mg/g | | 123.1 mg/g | 126 mg/g |
| Elektra | 0.5-1.15% | ND | 19.4-23.28% | 20-25% |
| | | | 194-232.8 mg/g | 200-250 mg/g |
| Lifter | 0.363% | ND | 18.96% | 22% |
| | 3.63 mg/g | | 189.6 mg/g | 223 mg/g |
| Harlequin | 7-15% | ND | 10-15% | 17-30% |
| | 70-150 mg/g | | 100-150% | 170-300 mg/g |
| Ringo's | 0.77% | ND | 14.25% | 15% |
| gift | 7.7 mg/g | | 142.5 mg/g | 150 mg/g |
| OG | 5-15% | ND | 10-15% | 15-30% |
| Kush | 50-150 mg/g | | 100-150 mg/g | 150-300 mg/g |
| Cannatonic | 5.25% | 0.15% | 12.73% | 18% |
| | 52.5 mg/g | 1.5 mg/g | 127.3 mg/g | 182 mg/g |

*THCA and CBCA are predicted to not be detected in genetically modified plants.
**Hypothetical values provided reflect the sum of THCA and CBDA measured previously in untransformed Cannabis; this prediction assumes the entire THCA production is transitioned to CBDA production.

The motif target sequence (SEQ ID NOs: 3-29) identified in Example 1 is used to engineer guide RNAs (gRNAs) for CRISPR/cas9-mediated gene disruption. gRNAs are designed with a guide sequence (or gRNA protospacer) having a 5′-end specific for the target sequence and a standard 3′-end stem loop that binds Cas9. The gRNAs are used to modify THCAS and/or CBCAS, or CBDAS.

### Example 4: Modified Plants

Multiple hemp cultivars are used to develop and optimize nanoparticle- or Agrobacterium-based transformation, tissue culture, and regeneration. Optimized approaches for deriving distinct hemp cultivars are determined by employing a series of distinct combinations of growth hormones and tissue culture/regeneration protocols (e.g., water status, temperature, and lighting). Transformation optimization is confirmed via the insertion of a green fluorescent protein (GFP) marker gene, and gene editing protocol optimization is confirmed by knockout of the phytoene desaturase (PDS) gene to produce albino seedlings.

Transient assays using agroinfiltration are used to validate the effectiveness of hpRNA constructs and gRNAs to inhibit target gene expression in vivo, using the protocol described by Deguchi et al., Sci. Rep. 10:3504, 2020. Standard stable in planta transformation techniques routinely utilized in other crops are leveraged as alternatives to the in vitro regeneration process (e.g., carbon nanotube delivery or ectopic meristem induction using developmental regulatory genes) to accelerate the regeneration process.

Following optimization of transformation, regeneration protocols, and gene editing, regeneration of stable transformants and molecular characterization of the transformed progeny are performed to confirm gene modifications. After generation of transformed seedlings, polymerase chain reaction (PCR) is used to initially confirm successful transformants, and then next generation sequencing is performed to validate the desired mutations at each target gene and/or hpRNA construct insertion. Validated transformant lines are allowed to mature, then resulting second generation plants are screened for a null-Cas9 segregant while fixing the target mutations in the homozygous state. Finally, the production of CBC, CBD, and THC is determined by High Performance Liquid Chromatography (HPLC) evaluation of third generation offspring of second-generation homozygotes.

### Example 5: Silencing CBDAS to Increase THC/CBC Expression

The single copy of CBDAS (ORF of SEQ ID NO: 123) identified in Example 1 can be used as a target to suppress all CBDAS expression, thereby providing plants that have an increased/enhanced level of THC and/or CBC.

Similarly to the system described in Example 1, the extremely highly conserved motif in CBDAS (shown in SEQ ID NO: 103) is used to generate hpRNA-encoding constructs for RNAi silencing of CBDAS in Cannabis, resulting in a stable and significant reduction of CBDAS activity. They were designed with a polynucleotide encoding an hpRNA with a stem targeting the CBDAS sequence, SEQ ID NO: 103. The polynucleotide is operably linked downstream to a C. sativa U6 promoter (e.g., SEQ ID NO: 139) to form a construct. This construct is useful for inactivating CBDAS in all tested Cannabis species and subspecies, because SEQ ID NO: 103 alone is sufficient to target CBDAS across myriad Cannabis plants.

This rationally created hpRNA that includes the highly conserved CBDAS sequence motif (SEQ ID NO: 103) is used to generate siRNA that hybridize with the mRNA transcripts created from the CBDAS gene, thereby inhibiting its expression. The plants are transformed and modified, similarly as described in Examples 3 and 4. It is predicted that the resultant increase in THCA production would assume all CBDA production. The resultant Cannabis plants exhibit increased THC and/or CBC levels.

1-24. (canceled)

25. A nucleic acid molecule comprising at least one polynucleotide operably linked to a plant promoter that functions in a Cannabis plant, wherein the polynucleotide encodes a hairpin RNA (hpRNA) molecule, and wherein the hpRNA molecule comprises:
a first nucleotide sequence encoding a first polyribonucleotide and being between 20 and 30 nucleotides in length which is substantially identical to the complement or reverse complement of a Cannabis THCAS/CBCAS gene of SEQ ID NO: 1, and
a second nucleotide sequence encoding a sense polyribonucleotide in the hpRNA molecule that is substantially the reverse complement of the first nucleotide sequence,
wherein the first and second nucleotide sequence are separated in the polynucleotide by a nucleotide sequence that encodes a loop structure in the hpRNA molecule.

26. The nucleic acid molecule of claim 25, wherein the first nucleotide sequence encoding a first polyribonucleotide and being between 20 and 30 nucleotides in length which is substantially identical to the complement or reverse complement of a Cannabis THCAS/CBCAS gene comprises at least 12 contiguous nucleotides of the complement or reverse complement of SEQ ID NO: 1.

27. The nucleic acid molecule of claim 25, wherein the first nucleotide sequence encoding a first polyribonucleotide and being between 20 and 30 nucleotides in length which is substantially identical to the complement or reverse complement of a Cannabis THCAS/CBCAS gene comprises at least 12 contiguous nucleotides of the complement or reverse complement of SEQ ID NO: 17.

28. The nucleic acid molecule of claim 25, wherein the first nucleotide sequence encoding a first polyribonucleotide and being between 20 and 30 nucleotides in length which is substantially identical to the complement or reverse complement of a Cannabis THCAS/CBCAS gene comprises at least 12 contiguous nucleotides of the complement or reverse complement of SEQ ID NO: 28.

29. The nucleic acid molecule of claim 25, wherein the polynucleotide encodes a hairpin RNA (hpRNA) molecule selected from the group consisting of SEQ ID NO: 128, SEQ ID NO: 129, and SEQ ID NO: 130.

30. The nucleic acid molecule of claim 25, wherein the polynucleotide does not comprise 8 or more contiguous nucleotides of the complement or reverse complement of SEQ ID NO: 103.

31. A vector configured to express the nucleic acid molecule of claim 25.

32. A genetically modified Cannabis plant material or commodity product made of or from a Cannabis plant comprising the nucleic acid molecule of claim 25 or the hpRNA molecule encoded by the polynucleotide.

33. The genetically modified Cannabis plant material or commodity product made of or from a Cannabis plant of claim 32, wherein the Cannabis plant is C. sativa, C. indica (non-hybrid), a C. sativa/C. indica hybrid, or C. sativa subspecies, C. ruderalis.

34. The genetically modified Cannabis plant material or commodity product made of or from a Cannabis plant of claim 32, wherein the plant material is a transgenic Cannabis plant comprising the polynucleotide stably integrated in its genome operably linked to the promoter.

35. A cell derived from the genetically modified Cannabis plant of claim 32, which cell comprises the hpRNA molecule or the hpRNA molecule encoded by the polynucleotide.

36. A method of making a genetically modified Cannabis plant, comprising:
transforming a cell of a Cannabis plant with the vector of claim 31 to produce a transformed cell;
regenerating a plant from the transformed cell, which plant is the genetically modified Cannabis plant.

37. A nucleic acid molecule comprising at least one polynucleotide operably linked to a plant promoter that functions in a Cannabis plant, wherein the polynucleotide encodes a hairpin RNA (hpRNA) molecule, and wherein the hpRNA molecule comprises:
a first nucleotide sequence encoding a first polyribonucleotide and being between 20 and 30 nucleotides in length which is substantially identical to the complement or reverse complement of a Cannabis CBDAS gene of SEQ ID NO: 103,
a second nucleotide sequence encoding a sense polyribonucleotide in the hpRNA molecule that is substantially the reverse complement of the first nucleotide sequence,
wherein the first and second nucleotide sequence are separated in the polynucleotide by a nucleotide sequence that encodes a loop structure in the hpRNA molecule.

38. A vector configured to express the nucleic acid molecule of claim 37.

39. A genetically modified Cannabis plant material or commodity product made of or from a Cannabis plant comprising the nucleic acid molecule of claim 37 or the hpRNA molecule encoded by the polynucleotide.

40. The genetically modified Cannabis plant material or commodity product made of or from a Cannabis plant of claim 39, wherein the Cannabis plant is C. sativa, C. indica (non-hybrid), a C. sativa/C. indica hybrid, or C. sativa subspecies, C. ruderalis.

41. The genetically modified Cannabis plant material or commodity product made of or from a Cannabis plant of claim 39, wherein the plant material is a transgenic Cannabis plant comprising the polynucleotide stably integrated in its genome operably linked to the promoter.

42. A cell derived from the genetically modified Cannabis plant of claim 39, which cell comprises the hpRNA molecule or the hpRNA molecule encoded by the polynucleotide.

43. A method of making a genetically modified Cannabis plant, comprising:
transforming a cell of a Cannabis plant with the vector of claim 38 to produce a transformed cell;
regenerating a plant from the transformed cell, which plant is the genetically modified Cannabis plant.
